(19) United States
(12) Patent Application Publication
(10) Pub. No.: US 2023/0174970 A1
(43) Pub. Date: Jun. 8, 2023

(54) Spatial multiomics and live biology with continuous imaging

(71) Applicant: Ramona Optics Inc., Durham, NC (US)

(72) Inventors: Natalie Alvarez, Durham, NC (US); Aurelien Begue, Durham, NC (US); Mark Harfouche, Durham, NC (US); Gregor Horstmeyer, Durham, NC (US); Robert Horstmeyer, Palo Alto, CA (US)

(21) Appl. No.: 18/074,431

(22) Filed: Dec. 2, 2022

Related U.S. Application Data
(60) Provisional application No. 63/285,585, filed on Dec. 3, 2021.

(51) Int. Cl. C12N 15/10; G01N 15/14

(52) U.S. Cl. CPC C12N 15/1065; G01N 15/1434; G01N 2015/0065

(57) ABSTRACT

An imaging system having multiple cameras providing a large field of view with sufficient resolution can be used for tracking movements of cells from their positions in a tissue sample into multiple isolated areas such as into individual microwells in a well plate. By determining the beginning and the end of the movements of each cell, the imaging system can associate the microwell locations to the original cell positions in the sample. Together with an analysis of the cells in the microwells, either individually or together with barcode beads, the analysis can achieve the spatial information needed for constructing a map of the molecular information with respect to the positions of the cells in the sample.

The present patent applicant claims priority from the U.S. Provisional Patent Application, Ser. No. 63/285,585, filed on December, 2021, entitled “Spatial multiomics and live biology with continuous imaging”, of the same inventors, hereby incorporated by reference in its entirety.

### BACKGROUND OF THE INVENTION

Spatial biology technologies obtain molecular data (DNA, RNA, Epi-DNA, Protein), cellular data (shape, size, whether the cell is alive or dead) and positional data (relative location) of cells in organized tissue structures. A common subset of spatial biology, spatial transcriptomics, aims to measure gene expression (presence and quantity of RNA transcripts) of each cell in a tissue sample and simultaneously measure the layout and orientation of the cells from a specimen. The field of spatial transcriptomics builds on single-cell sequencing (sc-seq), which enabled scientists to associate RNA information to individual cells as opposed to groups of cells in bulk sequencing.

Spatial biology helps scientists map the spatial architecture of a cell and how it talks to and interacts with its surroundings. This is important in neuroscience, oncology, immunology, and developmental biology—for example, in helping answer questions in oncology, “What are the cells around the tumor doing? Why is that cell able to attack and destroy cancerous cells, but not the other cells? Why was the therapy effective on this population of cells, but not that other population?”

To answer these questions, scientists benefit from knowing the relative position of each cell and its molecular profile:

Genes→genomics

Gene regulation→epi-genomics

Gene expression (mRNA)→transcriptomics

Proteins present→proteomics

Together, this information is referred to as the “omics” of a cell. Multi-modal omics or “multiomics” is the ability to measure the multiple molecular aspects listed above. Although the Holy Grail is to measure location, DNA, RNA, and protein in each cell, it is currently not feasible since measurement of one metric often compromises the other metric. Scientists are working to advance simultaneous multi-modal omics, but it is heavily driven by the biological limitations or compromises. As a result, multiple experiments are required and if possible, copies of cells. Commonly, spatial biology methods provide location information and either transcriptomic or proteomic data.

Spatial biology is a relatively new field. Spatial biology methods that provide spatial-omics data are used exclusively in research settings—in both academia and industry/pharma. Spatial-omics data may eventually inform personalized medicine and treatments, for example, to identify therapies targeted to the cell types and hierarchies or layout identified in the tumor microenvironment of a patient. Spatial biology may become a critical component in diagnosis—eventually, a merging of pathology and molecular laboratory testing.

A main area of focus for omics studies is transcriptomics (gene expression) because this varies significantly from cell to cell—it's what makes cells different in an organism since the genomics of the cells are the same (mostly). The field of spatial transcriptomics is quickly advancing and there are many groups developing technologies to move the field forward. These technologies stem from two camps of thought:

Camp 1. Assess entire genome. Characteristics of this camp include looking for presence of any and all RNA transcripts, e.g., untargeted or unbiased search, together with limited quantification (counting individual transcripts), e.g., good for presence Yes/No.

Camp 2. Assess limited number of specific genes. Characteristics of this camp include looking to quantify expression of specific transcripts, e.g., targeted or biased search, together with good quantification (counting individual transcripts), e.g., good for total expression level.

For the two general camps described above—entire genome (unbiased) and subset of genome (biased)—there are four main methodologies used for the assessment. In camp 1 for genome wide and unbiased search, the method includes physical isolation, for example, laser cut of Region of Interest (“ROI”), with high control and experiment customization, but tedious and very low throughput. The method also includes captured based, for example, permeabilization of tissue to extract RNA, with high throughput or lots of cells for all (20,000) genes, but single-cell resolution not guaranteed and with poor quantification.

In camp 2 for limited genes and biased search, the method includes in situ imaging, for example, targeted probes and imaging, and in situ sequencing, for example, rolling amplification of RNA and imaging, both with cellular and sub-cellular resolution and good quantification, but slow and long imaging time with limited number of probes (100-1,000 out of 20,000 genes).

A summary of the numerous technologies for these spatial transcriptomics approaches is provided in Table 1. The methods are organized in terms of ascending processing speed. For additional context, an overview of current single-cell RNA sequencing (sc-RNA-seq) technologies are provided in Table 2. These technologies do not retain spatial information about the sample.

Current solutions for single cell analysis without spatial information

An advancing solution to developing molecular data can be observed in U.S. Pat. No. 10,954,570, “Massively Parallel Single Cell Analysis” by Fan et al., hereby incorporated by reference in its entirety, describes a system for analyzing molecular data from thousands to millions of cells in a single event. This patent formed the basis of the technology developed by Beckton, Dickinson and Company released as BD Rhapsody™. Common analytic techniques separate cells using enzymes and mechanical agitation with the resultant fluid being cells and enzymes. This fluid has the enzymes deactivated and the fluid is then centrifuged to concentrate the cells. The cells are then suspended in a fluid for use as a carrier and the fluid and cells are spread on a plate with wells. The cells sink into the wells in the plate. Well plates historically had 96 wells but have increased by doubling, tripling or quadrupling that number with well size decreasing. It is now common to see hundreds of thousands, if not millions of wells, each on the order of 30 um-50 um in diameter on a single plate. These high-count well plates can be manufactured using photolithographic and polymer molding techniques that are derived from the semiconductor manufacturing sector. These high-count wells are commonly referred to as microwell arrays (referring to the well diameter) or nanowell arrays (referring to the volume of liquid that each well can hold). In single cell-sequencing workflows, each miniature well is designed to isolate and contain a single cell. The approaches with cells isolated in microwells are referred to as “well-based”.

Alternatively, single-cells can be isolated in miniature oil droplets in a flow-based workflow. The Chromium platform by 10×Genomics builds on the Drop-seq technology created in the lab of Steve McCarroll at Harvard. The approaches with cells isolated in oil droplets are referred to as “droplet-based”.

Both well-based and droplet-based single-cell isolation workflows require co-isolation with a synthetic molecular barcode. These barcodes are often in the form of oligonucleotide strands that bind with cellular RNA. U.S. Pat. No. 10,246,703, hereby incorporated by reference in its entirety, provides an overview of one common barcoding approach. Barcodes co-isolated with individual cells may be used to provide a unique identifier to the molecular contents of the cell (after lysing the cell to release the contents into the microwell or droplet). The barcode and molecular contents (ex. RNA) are then processed with downstream chemistry before going into a conventional next-generation sequencing workflow, for example, Illumina.

A key tenant of single-cell sequencing—both well-based and droplet-based is the need to isolate single cells for analysis. Poisson statistics is used to maximize the number of wells or droplets that contain 1 cell, and minimize the number of wells or droplets that contain 2 or 3 cells. Having multiple cells in isolation contaminates the data because many more molecules are then detected “per well” in downstream analysis. As a result of the Poisson loading distribution, many wells and droplets will necessarily be empty. Most literature refers to an overall ratio of about 10:1 (wells:cells).

Current commercial technologies for spatial biology

As of 2021, numerous companies have developed or are developing tools and systems that allow researchers to obtain molecular data on a cell and correlate the original location of that cell in a specimen such that the cell's prior relative location to other cells in the specimen can be determined.

1. Akoya Biosciences: Akoya has commercialized an approach developed at the Nolan Lab in Stanford which allows the user to stain the specimen with up to 40 pre-selected antibodies each with a fluorescent marker. The fluorophores are read three colors at a time and washed off before the next reading. This allows individual cells in a specimen to be associated with an antibody while retaining their location in the specimen. This limits the user to pre-selected antibodies. This technology is essentially standard immuno-histo-chemistry on steroids. It provides information about the proteins present with a limited and targeted number of probes. It does not directly provide information about RNA and DNA in the cells.

2. 10× Genomics: 10× Genomics' Visium Spatial Gene Expression product integrates spatial information from traditional histology (slide staining to see morphology and proteins) with high-throughput RNA sequencing to generate whole transcriptome gene expression profiles for frozen or formalin-fixed paraffin embedded (FFPE) tissue samples or targeted gene expression profiles (only possible with fresh frozen samples). For the Visium, each capture area (6.5×6.5 mm) contains 5,000 barcoded spots that are 55 μm in diameter (100 μm center to center between spots providing an average resolution of 1 to 10 cells). The tissue sections are stained and imaged with a bright field microscope and fluorescence to detect proteins. The specimen is then permeabilized so that cells release mRNA, which then binds to the spatially barcoded oligonucleotides. Reverse transcription leads to the production of cDNA, which is collected for downstream processing and library preparation. The library of barcodes is sequenced with an Illumina sequencer to determine the transcriptional profile of the tissue sections. The barcodes provide the spatial relationship to their original location on the slide and thus spatial relationships can be obtained along with the molecular data. The “resolution” being on the order of the spot size is more or less 55 to 100 microns. 10× Genomics is releasing an HD version of Visium in early '22 with a spot size of 4 microns (spaced at 10 um) to aim to get “single-cell resolution”.

10× Genomics also has a single-cell analysis platform called Chromium X, which has flexibility to be extremely high throughput with the goal of up to 1M cells.

3. NanoString Technologies: NanoString has developed a system using target oligonucleotides with fluorescent biomarkers. U.S. Pat. No. 10,501,777 “Simultaneous quantification of a plurality of proteins in a user-defined region of a cross-sectioned tissue” and U.S. Pat. No. 10,640,816 “Simultaneous quantification of gene expression in a user-defined region of a cross-sectioned tissue”, hereby incorporated by reference in their entirety, are relevant. These are conceptually similar but one patent is for analyzing proteins and the other for analyzing RNA. The oligo and fluorescent biomarker can be cleaved from the RNA or antibody using UV light and then collected and analyzed. An extremely small and accurate UV laser that has its illumination location in the specimen known is used to detach the biomarker such that a region of interest or a single cell can have its biomarker photo-cleaved and then analyzed. The data from the biomarker can be associated with an exact location in the specimen. This system is slow and currently limited to a relatively small number of samples being photo-cleaved from the specimen. As with the Akoya solution, a known set of targets must be chosen for use in attaching to the specimen RNA or protein.

4. Rebus Biosystems, Resolve Biosciences, ReadCoor, CARTANA: these are other companies developing high-resolution in-situ imaging based approaches (similar to Nanostring), where a high-resolution microscope (40×-60×) is used with reagents for direct imaging of fluorescence probes, or direct imaging of amplified RNA nucleotide sequences.

5. Laser Dissection: Single cell laser dissection can be used to separate and capture a single cell or a group of cells from a specimen. These samples may then be operated upon in a variety of known methods to detect molecular data. Since the dissection location in the specimen can be known for each sample, then molecular data and associated spatial information can be obtained. This is a general solution but so far limited in the number of cells that can be collected efficiently.

The problem can be summarized as there is no system that can do genome-wide (untargeted) assessment of gene expression with adequate sensitivity for quantitative studies while providing spatial context.

As quoted by Lundeberg: “In-situ capture avoids the typical limitations of direct visualization and allows for an unbiased analysis of the complete transcriptome. However, the main hurdle for these methods is restricted RNA capture efficiency, which becomes increasingly more challenging with higher resolution (i.e., smaller capture/barcoded areas)”

And as quoted by Waylen et al.: “High-resolution, imaging-based approaches require high magnification and fine sectioning necessitating long imaging times, which will protract further with scaling. Methods which employ targeted probes carry the inherent limitation of finite available fluorophores, and will be challenging to scale. Underpinning these challenges is that, as more cells and tissues are analyzed with higher resolution, more data points are required, demanding higher computational power, and scalable mathematical models for future 3D visualization and data interpretation”.

### SUMMARY OF THE EMBODIMENTS

In some embodiments, the present invention discloses a multi-aperture microscope technology that offers the ability to track in real-time and image multiple independent small model organisms, or other small items, such as cells, over a large area. The technology includes an organized array of multiple micro-cameras incorporated in a microscope to provide a large field of view with sufficient resolution for tracking movements of cells from their positions in a tissue sample into multiple isolated areas such as into individual microwells in a well plate. For example, a processor coupled to the multiple cameras can be used to run an organism tracking algorithm, which is able to compute position coordinates over time of each cell from a sample, to obtain the beginning positions of the cells in a sample to the end position of the cells at individually isolated areas.

By determining the beginning and the end of the movements of each cell, the imaging system can associate the microwell locations to the original cell positions in the sample. Together with an analysis of the cells in the microwells, either individually or together with barcode beads, the analysis can achieve the spatial information needed for constructing a map of the molecular or cellular information with respect to the positions of the cells in the sample.

In some embodiments, the present invention relates to mechanisms, chemicals, software and processes that are utilized to obtain molecular or cellular information of single cells while maintaining knowledge of the spatial origin of each cell from a specimen. The invention seeks to bridge the benefits of single-cell sequencing (an unbiased approach to detect and quantify expression levels of all possible genes in each cell, with a relatively high level of sensitivity at sufficient speed to fit into existing workflows) and the benefits of single-cell spatial context (understanding how individual cells are spatially organized in tissue and interact with one another). This invention accomplishes this by using a multi-camera array microscope (MCAM) to observe a large area continuously (synchronous and continuous information in space and time) so that the cells or components from a specimen can be uniquely identified as they are isolated and analyzed. The process maintains the viability of cells to enable researchers to conduct assays such as studying the response of cells to stimuli such as therapies.

The system tracks each cell from its initial location in the sample and follows each cell as they travel to the well plate locations. Once in the well plate the cells are analyzed and data is generated on each cell such as molecular (DNA, RNA, protein information) with the data associated with each individual cell. The molecular data is then associated with the cell initial locations in a sample image.

In some embodiments, the cells from the individual cells can be grouped together for a faster analysis. Distinction between cells from the group of cells can be accomplished by the use of barcoding technology, such as barcode beads introduced to each well of the well plate. The analysis results can provide the barcode information together with the molecular information, which can enable the sorting of the molecular information based on the barcode information.

In some embodiments, the barcodes from the barcode beads can be characterized by a fluorescence analysis, such as a sequential 4-channel fluorescence process to identify fluorescence signals emitted by the barcodes.

In some embodiments, the microscope can be equipped with fluorescence imaging capability, such as providing the cameras and the light sources with appropriate fluorescence filters, with the light sources emitting light through fluorescence excitation filters and the cameras capturing images through corresponding fluorescence filters. The fluorescence imaging capability can allow the microscope to characterize in-situ the barcode beads in each isolate well of the well plate, for example, through a sequential four-channel fluorescence imaging process at the well plate after introducing the barcode beads to the well plate.

### BRIEF DESCRIPTION OF THE DRAWINGS

Table 1 shows spatial transcriptomic technologies and performance.

Table 2 shows single cell sequencing technologies and performance.

Table 3 show a value proposition for the present invention.

FIG. 1 illustrates a process schematic for a spatial biology process according to some embodiments.

FIGS. 2A-2B illustrate flow charts for methods of spatial biology according to some embodiments.

FIGS. 3A-3B illustrate a schematic of an MCAM system according to some embodiments.

FIGS. 4A-4B illustrate configurations for an MCAM according to some embodiments.

FIGS. 5A-5D illustrate MCAM configurations according to some embodiments.

FIGS. 6A-6C illustrate configurations of an imaging system configured for 2 or 3 dimensional movement tracking according to some embodiments.

FIGS. 7A-7B illustrate flow charts for forming an assembly fixture configured for dissociating and for transporting cells in a sample into isolated areas according to some embodiments.

FIGS. 8A-8B illustrate flow charts for forming an imaging system configured for tracking movements of cells from a sample into isolated areas according to some embodiments.

FIGS. 9A-9C illustrate a process for a spatial construction of molecular information of cells in a sample according to some embodiments.

FIG. 10 illustrates a configuration of an assembly fixture for determining movements of cells according to some embodiments.

FIGS. 11A-11B illustrate a set up configuration for determining original positions of cells in a sample according to some embodiments.

FIGS. 12A-12G illustrate a process flow for forming a spatial map of a sample according to some embodiments.

FIGS. 13A-13B illustrate flow charts for a spatial construction of molecular information of cells in a sample according to some embodiments.

FIGS. 14A-14B illustrate a barcode bead configuration according to some embodiments.

FIGS. 15A-15B illustrate a barcode characterization according to some embodiments.

FIGS. 16A-16B illustrate filter configurations for barcode sequential fluorescence capture according to some embodiments.

FIGS. 17A-17G illustrate a spatial biology process using barcode beads for labeling cell content according to some embodiments.

FIGS. 18A-18C illustrate flow charts for a spatial construction of molecular information of cells in a sample according to some embodiments.

### DETAILED DESCRIPTION OF THE EMBODIMENTS

This invention seeks to bridge the benefits of single-cell sequencing (genome-wide, good sensitivity, high-throughput) and spatial biology (single-cell contextual resolution). This invention accomplishes this by using a novel multi-camera array microscope (MCAM) to observe a large area continuously so that the cells or components can be followed continuously across time and space. The position of all objects in the field-of-view can be traced through time and space by using software that uniquely tracks every individual particle.

The proposed invention also aims to keep cells alive throughout the process to enable downstream live biology—chemical screening, metabolic assessment, growth, replication, etc. Table 3 shows the overview of the system and bridging of benefits.

In some embodiments, the present invention aims to provide a solution that can (1) link the data from conventional and emerging single cell assay and molecular or cellular analysis techniques with the spatial location of any or all the cells and/or nuclei in a specimen's native environment, (2) accomplish this with a system that fits with the techniques and equipment (workflow) that is commonly used by researchers, and (3) enable assays or experiments to be conducted on live cells prior to downstream analyses such as sequencing.

In some embodiments, the present invention discloses a multi-aperture microscope technology that offers the ability to track in real-time and image multiple independent small model organisms, or other items, such as cells, over a large area. The technology includes an organized array of micro-cameras which, together, capture image data of a group of organisms distributed over a wide arena. A first processor (e.g., in the form of a field-programmable gate array (FPGA)) aggregates and streams video data from all micro-cameras simultaneously to a second processor (e.g., within a nearby desktop computer). It is then possible to run an organism tracking algorithm, which is able to compute per-organism position coordinates over time, to obtain the beginning positions of the organisms in a sample to the end position of the organisms at individually isolated areas. The technology can conduct cell tracking and imaging with no or minimum moving parts and is immune to the performance tradeoffs faced by other microscope technologies.

In some embodiments, the present invention discloses systems and methods for spatial biology, with related molecular or cellular data with relative locations of cells in organized tissue structures. The systems and methods include mechanisms, chemicals, software and processes utilized together to obtain molecular or cellular information from single cells or components of cells while maintaining knowledge of the spatial origin of each cell or component from a specimen.

The spatial origin of the cells can be obtained by using a multi-camera array microscope (MCAM) to observe a large area continuously (synchronous and continuous information in space and time) so that the cells or components from a specimen can be uniquely identified as they are isolated and analyzed. The MCAM can be configured to track each cell from its initial location in the sample and follow each cell as they travel to the well plate locations. The process maintains the viability of cells during the movement tracking to enable downstream live analysis to provide molecular or cellular information, which includes biology-chemical screening, metabolic assessment, growth, replication, or cell information. For example, once in the well plate, the cells are analyzed and data is generated on each cell such as molecular (DNA, RNA, protein) information or cellular information (shape, size of the cells, or whether the cell is alive or dead) and this data is associated with each individual cell. The molecular or cellular data is then associated with the cells initial location in a sample image.

In some embodiments, the present invention discloses systems and methods for spatial biology, which can be configured to provide molecular or cellular data, such as DNA, RNA, Epi-DNA, protein, size, shape, or cell characteristics, which correspond to positional data of cells in a sample. The systems and methods can also be used for spatial transcriptomics, which aim to measure gene expression, such as the presence and the quantity of RNA transcripts, of each cell in a tissue sample and simultaneously measure the layout and orientation of the cells from a specimen. The spatial data can assist in mapping the spatial architecture of a cell in a sample and how the cell interacts with its surroundings.

In some embodiments, the systems can perform genome-wide assessment of gene expression with adequate sensitivity for quantitative studies while providing spatial context. The methods can include a process to isolate single cells from the sample for analysis, e.g., for single-cell sequencing, for example, to isolate the cells in wells with at most one cell per well to avoid cross cell contamination in the analysis.

In some embodiments, the present invention discloses systems and methods configured to bridge the benefits of single-cell sequencing (an unbiased approach to detect and quantify expression levels of all possible genes in each cell, with a relatively high level of sensitivity at sufficient speed to fit into existing workflows) and the benefits of single-cell biology with spatial context (understanding how individual cells are spatially organized in tissue and interact with one another). The bridging accomplishment can be achieved by using a multi-camera array microscope (MCAM) having multiple cameras to provide a large combined field of view with sufficient resolution to track the individual cells continuously across time and space. The MCAM can be configured to keep the cells alive to enable downstream analysis, including conducting assays such as studying the response of cells to stimuli such as therapies. The MCAM can offer spatial information genome-wide at single cell level at a medium to high sensitivity and high throughput, together with downstream live cell analysis capability.

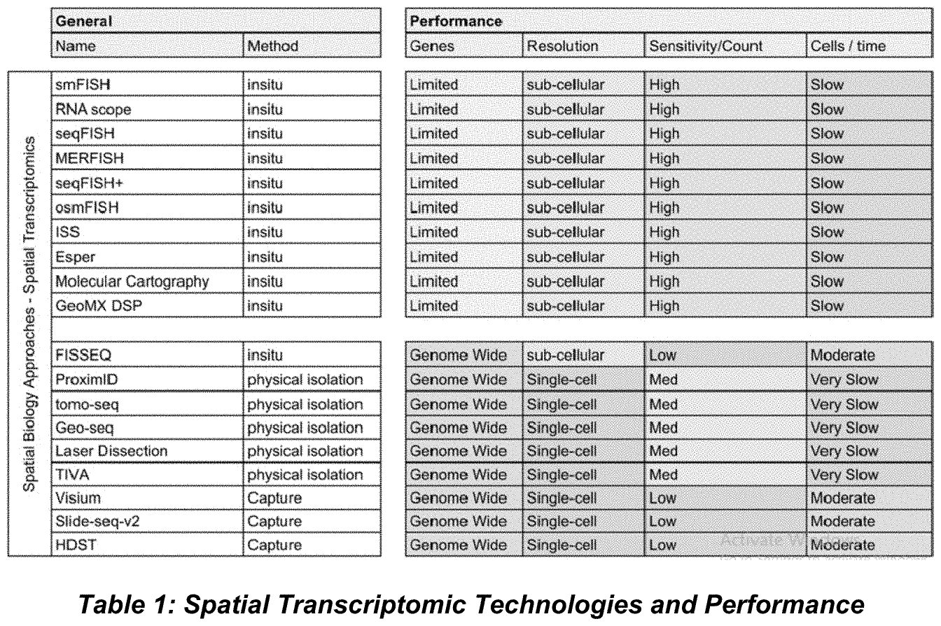

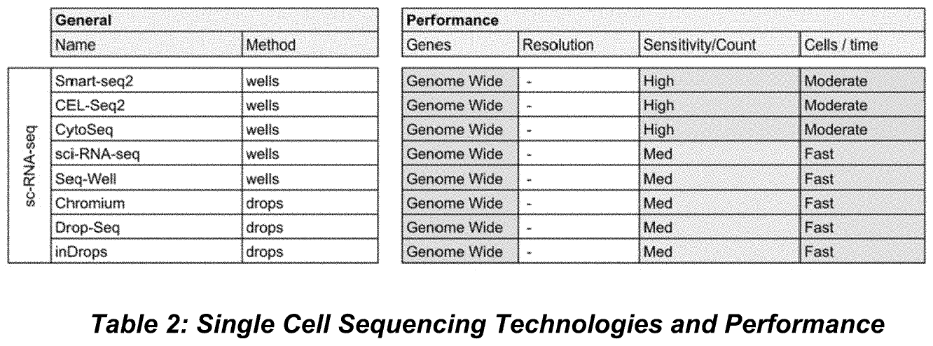

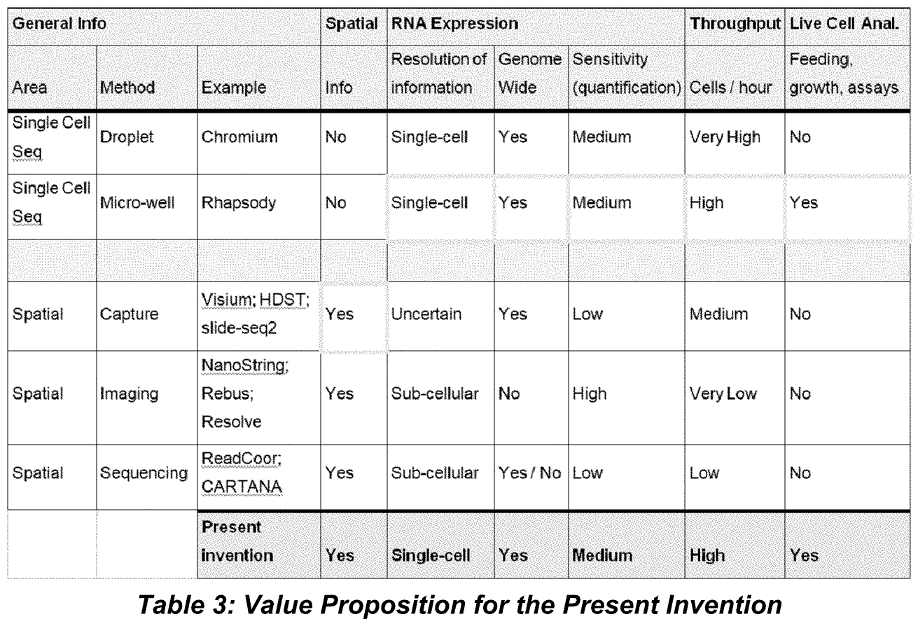

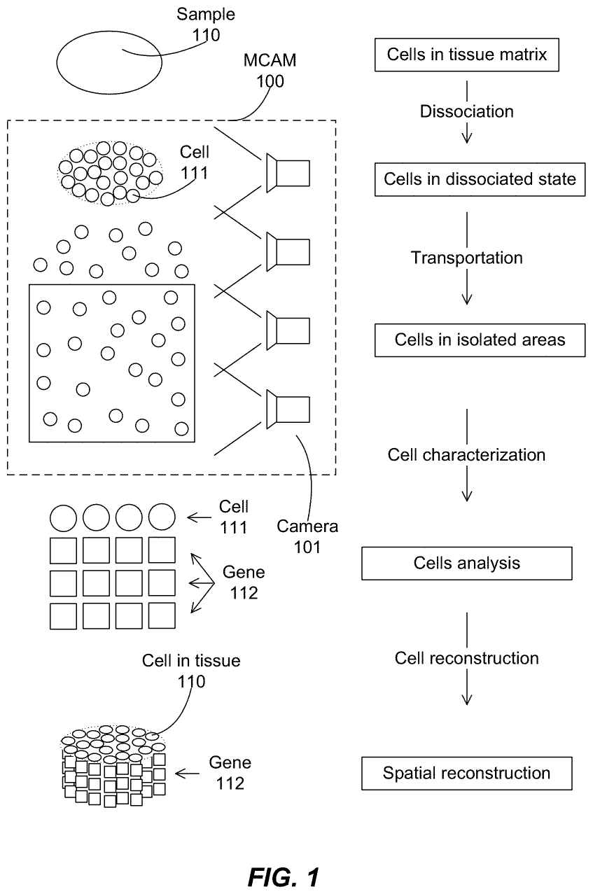

FIG. 1 illustrates a process schematic for a spatial biology process according to some embodiments. A sample 110 is prepared. The sample can be a tissue or organ, which has multiple same or different types of cells 111, such as red blood cells, white blood cells, or any type of cells. The sample can be taken from one or more subjects, which can be a human or non-human, living or dead, and healthy or diseased subject.

The sample 110 is mounted on a sample holder section of an assembly fixture disposed under a microscope such as an MCAM 100, which has multiple cameras 101 assembled together to provide a large field of view with high resolution. The sample can be first imaged at a high resolution to determine the location of the cells in the specimen using the microscope 100. The high resolution can be the highest image resolution offered by the microscope, e.g., without concern about the exposure time.

For example, at the start of the workflow, a tissue sample can be stained via immunohistochemistry techniques including chromogenic or immunofluorescence approaches to highlight morphological characteristics and to detect the presence and location of antigens (proteins). For example, a DAPI (4′,6-diamidino-2-phenylindole) is a blue-fluorescent DNA stain that exhibits about 20-fold enhancement of fluorescence upon binding to AT regions of dsDNA, which can be used on live cells to visualize the nucleus through fluorescence imaging.

The microscope can be used to acquire images of the stained tissue section, which can serve as a visual reference image of the sample/sections prior to downstream analyses. Initial microscope images for the specimen may be acquired at various optical resolutions as required by the application/user. Fiducial marks can also be used in the holder to provide location precision. Capturing the static starting/reference image of the sample is done prior to the singulation step in the process. Afterwards, the optical resolution of the system may then be changed to that which is required for continuous video observation over the entire field-of-view which is needed to track moving cells/particles.

The sample then undergoes a dissociation process, for example, by an enzyme introduced to the sample holder section of the assembly fixture. During the dissociation, the cells can be dissociated from the sample to be transported along a transport section to a section including multiple isolated areas, which can be a well plate containing multiple wells such as microwells.

As the process continues, the cells are dissociated and move in relation to each other and to their initial location on the sample holder. As the cells move, the microscope takes sequential images that are then compared using an object tracking algorithm to determine the path taken by each cell. The cell location can be associated with a Cartesian grid on the sample holder section to precisely identify their location. The cells can be dissociated into individual cells 111 or groups of cells and non-cellular matter of the sample. The cells can be moved by a fluid that flows the cells into individual isolated areas, e.g., individual wells on a well plate. Each cell is tracked as it moves from its original location in the specimen to a well in the well plate using the microscope.

The microscope can be used to track the motion and determine the location of all the cells in the sample during their dissociation and movement until they are finally located in separate wells in a well plate. The movement tracking can be used to find the positions of the cells in the sample by knowing their locations at the well plate, e.g., the locations of the wells that the cells are finally located. The movement tracking can be performed by the microscope at a video resolution, e.g., a lower resolution than the image resolution, to capture an image sequence showing movements of the cells. The video resolution is configured to provide fast image capture, such as at 30 fps, 60 fps, or any number of frames per second depending on the rate of dissociation and transportation of the cells. The video resolution is also configured to provide adequate resolution to identify cells, such as to identify the movements of cells when they cross paths.

For example, the microscope is configured to take sequential image frames of the entire field-of-view at frame rates sufficient to allow cell and particle tracking over the entire field-of-view, with resolution adequate to uniquely identify and differentiate cells at 5 micron sizes and even smaller components such as nuclei and other matter. The continuous tracking and unique identification of each particle allows for re-construction of the path of every particle in the scene such that the starting and ending position of every object can be uniquely assigned. Thus, the microscope can be used to monitor the movement of cells from their native environment in the tissue matrix, e.g., the sample, to the isolated wells, which can allow an association of the well locations in the well plate with the cell positions in the sample.

Once the cells are in the wells in the wells of a well plate, the cells can be examined, cultured, assayed, tested and manipulated using available techniques. Also, the RNA, DNA or protein in the cell can be obtained and manipulated by amplification or conversion of the RNA to cDNA for example. The well contents are then analyzed to generate cellular information or molecular information 112 using any of a variety of instruments such as cell counters or DNA sequencers. These devices produce data regarding the cells and in many situations molecular or cellular data from the cell is obtained.

In some embodiments, the cells are individually analyzed. In some embodiments, the cells are mixed together, for example for library preparation for single-cell analysis with next-generation sequencing. In this case, unique barcodes, typically attached to microbeads, can bind to the cell contents in each well to assign a unique molecular identifier to each cell. Each identity is unique and can be determined through sequencing by ligation or other methods, and deciphering the resulting sequential optical signal into a barcode that is unique to each cell/well. Once the contents of each cell in a well are bound with a unique barcode, the contents may be mixed/pooled and prepared for additional processing such as next generation sequencing. Once the barcode and molecular information of the cell contents are determined by next-generation-sequencing, this information can link each cell or object to its native starting position in the sample, based on a back tracking from the barcode data to the well location due to the unique identifier of the barcode in each well, and from the well location as the ending position of the cell or object to the starting position of the cell or object within the native environment due to the movements of the cell or object.

In some embodiments, the present spatial biology process can provide spatial transcriptomics at the single-cell level due to the physically isolated cells in physical isolated areas, e.g., separate wells. The method can be sure of single-cell resolution due to the elimination of diffusion of molecular contents across cells as they are lysed.

The present spatial biology process ensures single-cell resolution for wells containing one cell. When multiple cells are co-isolated in a well (multiplets), the database may be updated to show that the analysis results are from multiple cells. The number of cells within one well can be ascertained by an analysis of the movement tracking of the cells to the wells, or from the barcode information associated with the well.

In some embodiments, the present spatial biology process can provide integration of workflows and efficiency that can be faster and more integrated than current workflows for pathological analysis, single cell analysis, single cell sequencing, and spatial analyses. In biology, the higher speed of the analysis can offer better data accuracy. For example, enzymes can attack proteins and cells can get stressed over time, which can affect their genetic expression and skew results from single cell sequencing studies. The present spatial biology process can maintain cells in a way that reflects their native positioning, condition, and genetic profile as closely as possible.

In some embodiments, the present spatial biology process can provide high-throughput live biology with spatial and molecular or cellular information. By maintaining viable cells, users can run assays on the cells (screen, feed, grow, examine, etc.) prior to treatment for molecular testing, such as for single-cell sequencing.

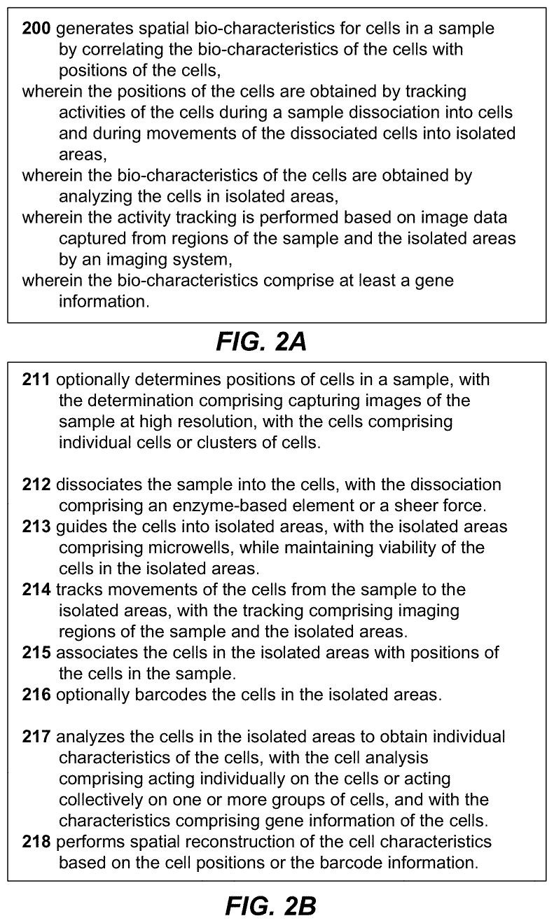

FIGS. 2A-2B illustrate flow charts for methods of spatial biology according to some embodiments. The methods can link the data from conventional and emerging single cell assay and molecular or cellular analysis techniques with the spatial location of any or all the cells and/or nuclei in a specimen's native environment, which can accomplish this with a system that fits with the techniques and equipment (workflow) that is commonly used by researchers, and which can enable assays or experiments to be conducted on live cells prior to downstream analyses such as sequencing.

In some embodiments, the systems and methods for spatial biology are configured to acquire a reference image of cellular or particle distributions in their starting or native position in the sample, and then track each cell from its initial location in the sample and follows each and every cell or particle as they travel to the discrete final locations, e.g., isolated areas such as isolated wells in a well plate. Once in their final location the cells are studied and analyzed. Data is generated on each cell regarding its response to assays for phenotypic information and molecular (such as DNA, RNA, or protein) or cellular (size, shape, dead or alive, or other characteristics) information. This data is associated with each individual cell. The data is then associated with the cell's initial location in the image of the sample as a cellular architecture (ex. tissue). In addition, the image data and cellular or molecular or cellular data can be visualized, such as forming a map or an image reconstruction of the sample with phenotypic and molecular or cellular information for each cell position.

In some embodiments, a system for spatial biology can include an imaging system which can capture images of a sample at a high resolution to form a reference for cells and cellular-size objects. The system can be configured to have a continuous field-of-view at a single-cell resolution at a video frame rate to monitor movements of the cells and cellular-sized objects. For example, the system can include multiple cameras having overlap fields of view to offer high resolution for a large combined field of view.

The system can be used with live human or mammalian tissue samples. The system can include radiation sources to image the sample with one or more illumination patterns, such as bright field or dark field. The radiation sources can provide radiation in the visible, infrared or ultraviolet ranges. Further, the radiation sources can be configured to provide fluorescence illumination modalities, such as having filters to allow fluorescence excitation signals to pass through. Correspondingly, the multiple cameras can be configured to capture fluorescence images, e.g., the fluorescence signals generated from the sample due to the fluorescence excitation signals. For example, the cameras can have filters to allow the fluorescence signals to pass through.

The system can be used with microfluidics, chemicals, or equipment that dissociates the tissue into singulated cells. The system can be used with a consumable that maintains cells and cellular-sized objects, such as in a monolayer within the field-of-view such that all of the objects are within the depth-of-field of the system and may be observed continuously across time and space.

The system can be used with software that tracks all of the objects, whether moving or not, in the field-of-view and uniquely identifies them throughout for the duration of the video capture. The system is configured to allow objects, e.g., cells, to flow into channels within the field-of-view and the objects' relative positions are known before being sent into downstream processes outside of the field-of-view, for example, for flow cytometry.

The system can be used in parallel with a protocol that maintains cells at temperatures and in conditions such that cells are viable/animated/alive for downstream assays and phenotyping (growth, replication, metabolic assessment, chemical screening, etc). The system can be equipped with a microwell array within the field-of-view that allows for isolation of individual cells within compartments. The system can be configured so that cells are co-isolated in wells with unique molecular identifiers, for example, oligonucleotide barcodes. The system can be configured to accept a lysis solution so that cells are lysed and analytes and unique molecular identifiers are extracted for downstream analysis. For example, the system can be used with in-situ sequencing methods such as imaged based sequencing-by-synthesis to optically differentiate nucleotide sequences between individual cells.

In some embodiments, the systems and methods can provide nucleic acid analysis of single cells of a sample with known positions from the sample. The methods can be used for massively parallel single cell sequencing, e.g., to analyze thousands of cells concurrently, with the thousands of cells being a mixed population of cells, e.g., cells of different types or subtypes, different sizes.

FIG. 2A shows a broad method for spatial biology. Operation 200 generates spatial bio-characteristics for cells in a sample by correlating the bio-characteristics of the cells with positions of the cells. The positions of the cells are obtained by tracking activities of the cells during a sample dissociation into cells and during movements of the dissociated cells into isolated areas. The bio-characteristics of the cells are obtained by analyzing the cells in isolated areas. The activity tracking is performed based on image data captured from regions of the sample and the isolated areas by an imaging system. The bio-characteristics comprise at least gene information.

FIG. 2B shows a more detailed method for spatial biology. Operation 211 optionally determines positions of cells in a sample, with the determination comprising capturing images of the sample at high resolution, with the cells comprising individual cells or clusters of cells. Operation 212 dissociates the sample into the cells, with the dissociation comprising an enzyme-based element or a sheer force. Operation 213 guides the cells into isolated areas, with the isolated areas comprising microwells, while maintaining viability of the cells in the isolated areas. Operation 214 tracks movements of the cells from the sample to the isolated areas, with the tracking comprising imaging regions of the sample and the isolated areas. Operation 215 associates the cells in the isolated areas with positions of the cells in the sample. Operation 216 optionally barcodes the cells in the isolated areas.

Operation 217 analyzes the cells in the isolated areas to obtain individual characteristics of the cells, with the cell analysis comprising acting individually on the cells or acting collectively on one or more groups of cells, and with the characteristics comprising gene information of the cells. Operation 218 performs spatial reconstruction of the cell characteristics based on the cell positions or the barcode information.

Micro-Camera Array Microscope (MCAM) System

In some embodiments, an imaging system can be used to track moving objects, such as cells from a dissociated tissue matrix, over a large imaging area and at high spatial resolution in real-time. The system is based upon an imaging hardware unit, computational hardware, and jointly designed software.

In some embodiments, the imaging system can include a microscope technology that offers the ability to track in real-time and image multiple independent small model organisms or other items, such as cells, over a large area, such as from a sample holder to multiple isolated areas. The technology can conduct small object tracking and imaging with little or no moving parts and is immune to the performance tradeoffs faced by other microscope technologies developed so far.

An array of more than one digital micro-camera, along with the use of patterned illumination and a digital post-processing operation, jointly create a multi-camera patterned illumination microscope (MCPI) or multi-camera array microscope (MCAM, used interchangeably with MCPI). Each micro-camera includes its own unique lens system and detector. The field-of-view of each micro-camera unit can partially overlap with the field-of-view of one or more other micro-camera units within the array. The entire field-of-view of a sample of interest is imaged by the entire array of micro-cameras in a single snapshot. All micro-cameras operate synchronously to obtain spatially continuous and temporally synchronous information across the entire field-of-view in every single frame. In addition, the MCAM system uses patterned optical illumination to improve its effective resolution. The MCAM system captures one or more images as the patterned optical illumination changes its distribution across space and/or angle at the sample. Then, the MCAM system digitally combines the acquired image sequence using a unique post-processing algorithm.

Specifically, the MCAM microscope can include multiple parallel image data acquisition devices, e.g., cameras, across an array of multiple separate image sensors and associated lenses, which can allow the image acquisition of a large sample, limited by the number of cameras in the camera array. The cameras can be micro cameras having small form factors assembled on a camera board, with a data transfer cable coupled to a nearby computer system. With the small size and short transfer cable, fast data acquisition for large samples can be achieved.

The cameras can be configured to have overlapped fields of view on the sample, which can allow stitching the images across neighbor cameras for a seamless view of the sample. The overlapped fields of view between adjacent cameras can be less than or equal to 50%, e.g., there are areas on the sample that can be imaged only by the middle cameras and not by the neighbor cameras. The overlapped fields of view can be 5%, 10%, 20%, 30%, or 40%.

The cameras can be configured to have 50% or more overlapped fields of view on the sample, which can allow sample depth analysis, in addition to the stitching ability, for example, through a photogrammetry process such as photometric stereo. For 50% or more overlapped fields of view, all areas on the sample can be imaged by at least two cameras, which can allow the depth analysis of organisms detected in the images, for example, through image disparity between the captured images for a same feature.

The microscope can include one or more light sources, which can be disposed above the sample, below the sample, or both above and below the sample. The light sources can be configured to provide one or more illumination patterns to the sample. For example, the light sources can be configured to provide bright field images or dark field images to the cameras. The multiple illumination patterns can also allow depth analysis through multiple images captured by a same camera but under multiple illumination patterns.

The microscope can be configured for fluorescence imaging, in addition to visible, UV or IR imaging. The fluorescence imaging capability can be accomplished by some of the light sources configured to generate fluorescence excitation signals, such as through fluorescence excitation filters for the light sources, and by some of the cameras configured to capture fluorescence signals, such as through fluorescence filters for the cameras. Sequential fluorescence imaging can also be performed, for example, by a moving filter assembly for changing filters for the cameras.

The microscope can include one or more moving mechanisms configured to move the individual cameras, the camera array, the light sources, or the sample. For example, each camera can have a sensor adjustment mechanism configured to move the image sensor of the camera, an optical lens adjustment mechanism configured to move the optical lens of the camera, an objective lens adjustment mechanism configured to move the objective lens of the camera, and a camera adjustment mechanism configured to move the camera. The camera array can be coupled to a stage moving mechanism configured to move the camera array with respect to the sample. The sample can be disposed on a sample support, which can be coupled to a support moving mechanism configured to move the sample support in one or more directions, such as in a direction toward or away from the cameras, or in directions parallel to the sample for repositioning the sample under the cameras, such as for scanning the sample.

The microscope can include one or more inputs configured to deliver a solution to an assembly fixture, which can affect the cells in a sample disposed in the assembly fixture, such as to provide an enzyme to the sample for dissociation or to deliver a lysis solution to isolated areas of the assembly fixture for exposing the contents of the cells in the isolated areas.

The microscope can include one or more processors configured to process the data from the images captured by the cameras. For example, the processors can be configured to run an organism tracking algorithm, which is able to compute per-organism position coordinates. The computational process can be performed on a main processor, or can be distributed across multiple processors.

The processors can include only a main processor, which can be configured to accept image data from the cameras, such as configured to serially accept the multiple parallel image data streams from the multiple cameras. The processors can include a pre-processor, such as a Field Programmable Gate Array (FPGA), in addition to the main processor. The pre-processor can be configured to accept the multiple parallel image streams from the cameras, to parallely process the multiple image streams, and then to serially send image data to the main processor for additional analysis. The processors can include multiple pre-processors, with each pre-processor coupled to a camera output for pre-processing the camera image data right after capturing the image. Outputs from the multiple processors can be sent serially to the main processor, for additional analysis. The conversion of multiple parallel data streams to a serial data stream can be performed by electronic devices, such as by an FPGA, which can aggregate and stream video data from all cameras or from all pre-processors coupled to the cameras simultaneously to the main processor, which can be a processor of a data processing system such as a nearby desktop computer.

The microscope can include a controller configured to control the cameras, the light sources, the input sources, and the moving mechanisms, for example, to set the image parameters for the cameras, the radiation parameters for the light sources, and the excitation parameters for the excitation sources. The controller can be configured to control the moving mechanisms for moving the cameras or the sample support, for example, to change the amount of the overlapped field of view between adjacent cameras. The controller can also be configured to accept inputs, such as external inputs from an operator or from a memory device, to provide camera parameters such as distances between cameras or the magnification of the cameras, light parameters such as the wavelengths of the light sources or the locations of the light sources with respect to the cameras, sample support parameters such as positions of the sample support relative to the cameras and the light sources. The controller can also be configured to accept inputs related to the organisms to be tracked, such as sizes and shapes of the organisms or possible types and identification of the organisms.

The controller can be configured to accept inputs related to the objects being tracked, with the inputs including at least object shapes, dimensions and characteristics, object types or object identification.

The controller can be configured to detect objects or partial objects in the captured images from individual cameras in 2 or 3 dimensions. The controller can be configured to merge or remove duplicate objects across neighbor cameras. The controller can be configured to determine characteristics to reject detected objects not meeting the input object data.

The controller can be configured to form bounding boxes and locations for the objects in 2 or 3 dimensions meeting the characteristics of the input object data. The controller can be configured to transform objects in bounding boxes. The controller can be configured to analyze the objects. The controller can be configured to form tracking data including movements of the objects.

In some embodiments, “controller”, “processor”, and “pre-processor” are electronic devices, and can be used interchangeably in the specification, with the distinction between these components based on the context. For example, pre-processor and processor can be the same device type, with a difference being the positions of the pre-processor, e.g., the pre-processor is configured to process data before sending to the processor for processing. An electronic device can be configured to function as a controller or a processor, e.g., a controller can be used to control devices, such as cameras, and at a same time, can be used to process data. A processor can be used to process data, and at a same time, can be used to control devices, such as cameras.

Thus, a controller can be a processor or a pre-processor, a processor can be a pre-processor or a controller, and a pre-processor can be a controller or a processor.

Unique properties of this tracking system include its ability to enable measurement of small model organisms, or items such as cells by imaging their behavior or motion at high resolution, in real-time, over a large field of view, and with no moving parts. The array of micro-cameras affords this technology multiple advantages over other tracking systems. Firstly, it expands the field of view of the system without sacrificing resolution. Second, it enables the tracking of multiple organisms or cells simultaneously, which other mechanical based tracking technologies generally cannot achieve. Third, it allows for full field of view imaging at full optical resolution but at low frame rates where the target organisms or cells and their surroundings are recorded as well as targeted imaging where only the tracked organisms or cells are visible and much higher acquisition frame-rates can be achieved. These features allow for a high level of versatility that enables a wide range of research and commercial applications.

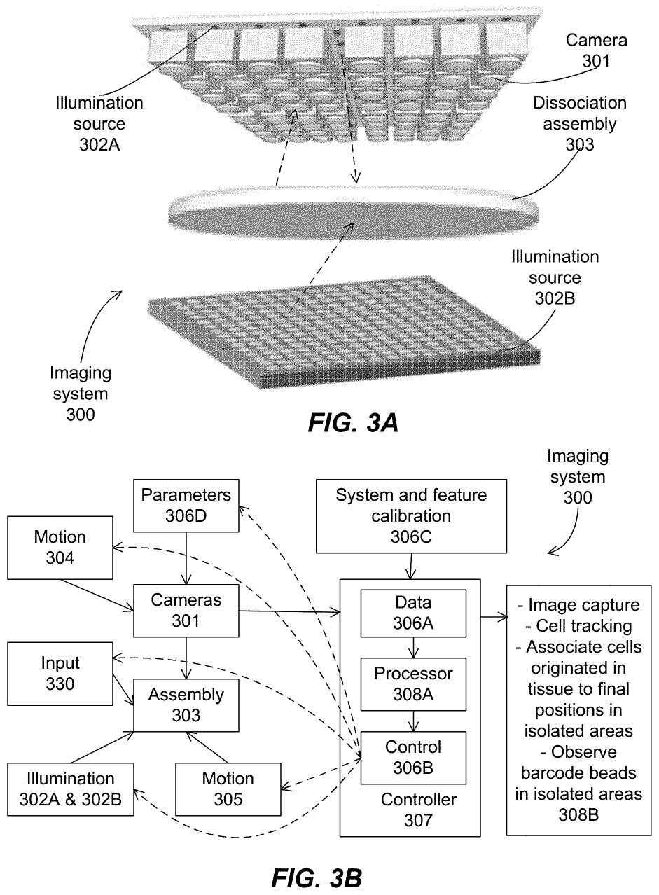

FIGS. 3A-3B illustrate a schematic of an MCAM system according to some embodiments. In general, the MCAM system can be viewed as an integration of multiple individual microscopes tiled together in an array to image a large sample. Each individual microscope can be configured into a micro camera package, e.g., a camera having a small form factor with minimum components, such as without a cover or extra peripheral elements. The integration of the micro camera packages can form a tightly packed array of micro-cameras with high resolution (1-10 μm) over a large area (hundreds of square centimeters). The images or video taken from the individual micro cameras, which include overlapped or non-overlapped image patches of a sample, can be assembled together to form the image of the complete sample. The MCAM system can offer size, weight, throughput, simplicity, and cost advantages with respect to standard microscopes. The MCAM system may not require any moving parts, and its micro-cameras fit within a compact space without requiring a rigid support structure and can thus operate within a small, confined space.

In FIG. 3A, the MCAM system 300 can include multiple cameras 301, which can form a camera array, and one or more illumination sources disposed above 302A and below 302B for microscopic imaging. The camera array can have a common clock generator to reduce timing variations between cameras. The cameras can include micro-camera packages, which can include multiple camera sensors and optical components assembled on a board, such as on a Printed Circuit Board (PCB). The light sources can be visible light sources, infrared light sources, ultraviolet light sources, fluorescent light sources, or polarized light sources, such as light emitting diodes (LEDs) or lasers with appropriate wavelengths and filters. The illumination system can be placed below 302B or above 302A the sample, to provide transmissive or reflective light to the micro cameras.

The MCAM system can use multiple micro-cameras 301 to capture light from multiple sample areas, with each micro camera capturing light from a sample area onto a digital image sensor, such as a charged coupled device (CCD), complementary metal-oxide semiconductor (CMOS) pixel array, or single-photon avalanche diode (SPAD) array.

In some embodiments, the illumination system can provide the sample with different illumination configurations, which can allow the micro cameras to capture images of the sample with light incident upon the sample at different angles, spatial patterns, and wavelengths. The illumination angle and wavelength are important degrees of freedom that impacts specimen feature appearance. For example, by slightly changing the incident illumination angle, a standard image can be converted from a bright field image into a phase-contrast-type image or a dark field image, where the intensity relationship between the specimen and background is completely reversed. The illumination system thus can be controlled to provide an optimum illumination pattern to the sample.

Alternatively, by providing the sample with different illumination light angles, spatial patterns, and wavelengths, both intensity and phase information of the imaged optical field can be recorded, which can allow the reconstruction of an image, for example, with more information or higher resolution, such as a measure of sample depth, spectral (e.g., color) properties, or the optical phase at the sample plane.

The MCAM system 300 can include a controller for controlling the cameras, the illumination sources, and for processing the images. For example, the cameras can have optional preprocess modules, which can be configured to preprocess the image data when reading from the image sensors of the cameras. The preprocess modules can perform simple or complex image processing, such as a quick detection of frame to frame variation or an object detection. The original or preprocessed image data can be sent, in multiple parallel data streams, to another optional process module, which is configured to organize the image data.

The process module can be an FPGA based module (e.g., a module containing a processing chipset, such as an FPGA, or other chipset of an ASIC, an ASSP, or a SOC), which can be configured to receive image data from the multiple camera units, e.g., through data streams. The FPGA based module can include a shallow buffer, for example, to store incoming data from the data streams. The FPGA based module can be configured to send sensor configuration data to the camera array, for example, to provide image parameters to the image sensors of the camera units. The sensor configuration can be received from a computational unit having a processor and a memory. For example, the processor can send configuration and settings to the FPGA based module, with the configuration and settings including setting information for the FPGA based module and the configurations for the image sensors. The FPGA based module can communicate with the computational unit using direct memory access (DMA) to pass data directly to the memory, through a high speed link such as PCIe. The FPGA based module can communicate with a control module, which can be configured to control lighting, motion, and sample handling for the microscope system. The computational unit can also communicate directly to the control module. The computational unit can communicate with a storage or network devices (not shown). The system can include peripheral devices, such as stages, illumination units, or other equipment involved in the apparatus necessary to ensure adequate imaging conditions.

The MCAM imaging system 300 can be used to record video of a sample of interest across a wide FOV and at high resolution. MCAM video is created by recording multiple image snapshots in sequence from one or more micro-cameras within the array.

FIG. 3B shows a block diagram of an imaging system 300, such as an MCAM system, modified for organism or cell detection and tracking. The imaging system can include a camera array 301 and an illumination source 302A and 302B, which are controlled by one or more controllers, such as a camera controller, an illumination controller, and a system controller.

An imaging system can include an array of cameras 301 focused on an assembly fixture 303 for supporting a sample under the illumination of an array of light sources 302A and 302B. Image parameters 306D to the camera array 301 can be inputted to the camera array, for example, to control focus mechanisms for focusing or for changing magnification of the individual cameras. A motion mechanism can include a movable camera stage 304, which can be used to adjust the positions of the camera array, such as tipping, tilting, translating the camera array, or for changing the overlap amounts between cameras. A motion mechanism can further include a movable sample holder 305, which can be used to adjust the positions of the sample, such as tipping, tilting, translating, or curving the sample. The movable sample holder can also be used for advancing the sample or the sample holder in discrete steps for capturing scanning image data of the sample. One or more inputs 330 can be used to provide chemicals to the sample in the assembly fixture 303, such as an enzyme solution or a lysis solution.

A data processing system 307 can be used to control the elements of the imaging system. The data processing system 307 can be configured to receive inputs 306C, such as data related to features of interest to be detected and analyzed on the sample. The data processing system 307 can be configured to receive data 306A from the camera array 301, and to transfer the data to a data processing processor 308A or 308B for processing. The data processing system 307 can be configured to transfer the data to a second data processing processor 308B for analysis. The data processing system 307 can include a controller 306B to control the camera array, the illumination source, and the sample holder to provide suitable conditions for image captures, such as providing variably illuminated radiation patterns to the sample, repositioning the cameras, the camera array, the sample, or the sample holder for focusing or scanning operations.

In some embodiments, the data processing system is a desktop computer. This desktop computer can be attached to a monitor for visual analysis of recorded MCAM video and/or MCAM statistics. The desktop computer can also be networked to transmit recorded video data and/or MCAM statistics and is also used to control the image and video acquisition parameters of the MCAM instrument (exposure time, frame rate, number of micro-cameras to record video from, etc.) via electronic signal.

The imaging system 300, such as a camera array microscope, based on a set of more than one compact, high-resolution imaging system, can efficiently acquire image data from across a large sample by recording optical information from different sample areas in parallel. When necessary, physically scanning the sample with respect to the array and acquiring a sequence of image snapshots can acquire additional image data.

In operation, after each of the cameras acquires an image, the image data from each camera are sent, in parallel to the FPGA. The FPGA then sequentially outputs the image data into a serial data stream to the processor to be processed, or to the memory of the processor. The parallel to serial conversion, e.g., in the FPGA, can be performed sequentially on each image or on portions of each image.

The imaging system can be used to obtain image and video data from the sample. The data can be analyzed to detect organisms or cells for tracking. An object detection algorithm, and subsequently, an object tracking and analyzing algorithm can be applied on the image data stored in the memory, including an edge detection algorithm, a projection algorithm, a centroid-finding algorithm, a neural network such as a convolutional neural network, or an inpainting algorithm. For example, the object detection is first performed to find the objects of interest, e.g., after removing the objects that are not suitable. The image data then can be cropped out to form bounding boxes, e.g., regions of interest. The bounding boxes can be centered upon each object of interest, and correlate specific objects as a function of time for tracking. Data from the bounding boxes are saved to the memory after processing.

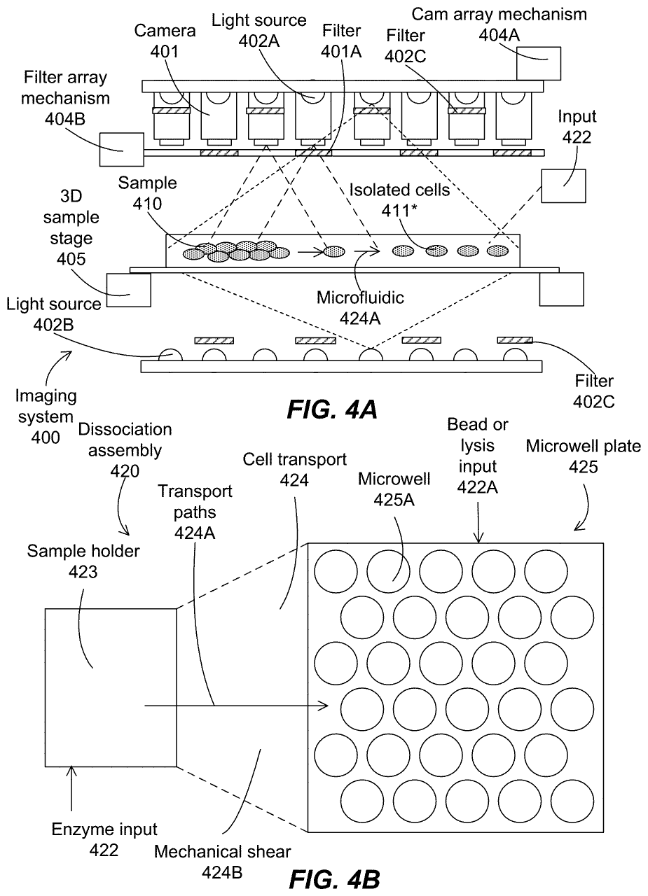

FIGS. 4A-4B illustrate configurations for an MCAM according to some embodiments. FIG. 4A shows a cross section view of an MCAM having multiple cameras 401 and one or more light sources 402A and/or 402B to illuminate a sample 410. The cameras and the light sources can be configured with or without filters, such as fluorescent filters or polarized filters. For example, as shown, alternate cameras and light sources have filters 401A and 402C, respectively. The filters for the cameras can change the characteristics of the captured light, so that the images captured by the cameras can have the specific property of the filters. For example, a fluorescent filter can allow the cameras to capture fluorescent signal emitted from the sample. A polarized filter, such as a circular polarized filter, can allow the cameras to capture circular-polarized light.

The filters for the light sources can change the characteristics of the emitted light, so that the sample can have the specific light property provided by the filters. For example, a fluorescent filter can allow the light sources to emit fluorescent excitation energy to the sample, causing the organisms in the sample to respond and emit fluorescent signals. A polarized filter, such as a circular polarized filter, can allow the light sources to emit circular-polarized light.

The MCAM system can include input sources 422 for provide a solution to the sample, such as an enzyme or a lysis solution.

The MCAM system can include moving mechanisms configured to move the cameras or the sample. A moving mechanism 404A can be coupled to the camera array to move the camera array relative to the sample, such as toward or away from the sample. Another moving mechanism 404B can be coupled to a filter array to move the filters for the cameras, for example, when performing sequential fluorescence imaging. Another moving mechanism 405 can be coupled to a sample support to move the sample relative to the cameras, such as toward or away from the cameras. The moving mechanism 405 can also be configured to move the sample support in a lateral direction, for example, for scanning the sample. For example, the specimen can also be placed on a 3D motorized stage, whose position can be controlled via software on the computer to bring the specimen into appropriate focus and lateral position.

Adjacent cameras can have different overlapping fields of view, such as more than 50% overlapped FOV, less than 50% overlapped FOV, or non overlapped FOV. Each camera has a field of view, which can depend on the camera magnification and the distance to the sample. Each camera can focus on a sample area, with non-overlapping area or overlapping areas with a nearby camera.

In some embodiments, the field of views of the cameras can be adjusted to vary the overlapping area, such as between non overlapping FOV, less than 50% overlapping FOV, and more than 50% FOV. The adjustment can be performed by changing the magnification of the cameras or the focus distance to the sample areas.

The FOV of the cameras can be non overlapped, for example, to observe samples with discrete areas such as well plates. The FOV of the cameras can overlap 50% or less in one or two lateral directions, such as x and y directions, such that less than half of the points on the object plane for one camera are also captured by one or more other cameras in the array. This permits stitching of the images to form a complete representation of the sample.

The FOV of the cameras can overlap 50% or more in one or two lateral directions, such that less than half of the points on the object plane for one camera are also captured by one or more other cameras in the array. This permits depth calculation for the objects positions, for example, through photogrammetry or photostereo.

FIG. 4B shows a top view of an assembly fixture 420 configured for dissociating a sample and for transporting cells in the sample to multiple isolated areas. The assembly fixture 420 can have a sample holder section 423, a section 425 for multiple isolated areas, such as having multiple isolated areas or having a support for supporting multiple isolated areas, and a cell transport section 424. The multiple isolated areas can be a microwell plate 425, which can have multiple micro wells 425A. The cell transport section 424 is coupled between the sample holder section 423 and the microwell plate section 425, with the cell transport section including transport paths 424A, such as microfluidic conduits, for cells. The assembly fixture can have an enzyme input 422, configured for accepting an enzyme to the sample holder for dissociating the sample. Other inputs 422A can be included, such as an input for barcode beads and an input for lysis solution.

The assembly fixture can be disposed in the MCAM, with the field of view of the MCAM covering the assembly fixture for observing movements of the cells in the sample to the isolated areas of microwells.

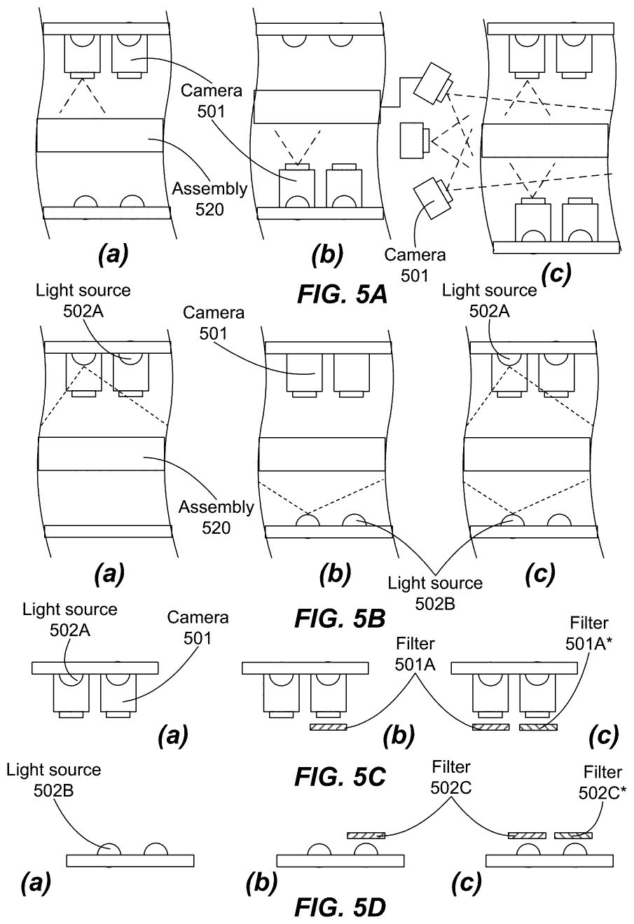

FIGS. 5A-5D illustrate MCAM configurations according to some embodiments. FIG. 5A shows configurations of the cameras or the camera array in an MCAM system. The cameras 501 can be disposed above the assembly fixture 520 in FIG. 5A(a), or below the assembly fixture in FIG. 5A(b). Other configurations can be used, such as the cameras can be disposed on a left side, on a right side, or forming an angle not parallel and not perpendicular with the assembly fixture (FIG. 5A(c)). Multiple cameras can be used, such as above the assembly fixture, below the assembly fixture, at a side of the assembly, or any combination thereof.

FIG. 5B shows configurations of the illumination sources or light sources in an MCAM system. The light sources 502A can be disposed above the assembly fixture 520 and at a same side as the cameras in FIG. 5B(a), to provide reflective illumination to the assembly fixture. The light sources 502B can be disposed below the assembly fixture and at an opposite side of the cameras in FIG. 5B(b), to provide transmissive illumination to the assembly fixture. The light sources 502A and 502B can be disposed above and below the assembly fixture, respectively, in FIG. 5B(c). Other configurations can be used, such as the cameras can be disposed on a left side, on a right side, or forming an angle above or below and not parallel and not perpendicular with the assembly fixture.

FIG. 5C shows filter configurations of the cameras or the camera array in an MCAM system. The cameras can have filters, such as fluorescent filter or polarized filters, to capture light with specific characteristics. The cameras can have no filters in FIG. 5C(a). Some cameras can have no filters and some cameras have filters 501A in FIG. 5C(b). The cameras can have multiple types of filters 501A and 501A* in FIG. 5C(c). Other configurations can be used, such as filtered cameras can be alternatingly arranged or randomly arranged with non-filtered or with different type filtered cameras. The filters can be fixedly coupled to the cameras. Alternatively, the filters can be movably coupled to the cameras, e.g., the filters are coupled to a filter array, which can move between different positions to provide different sets of filters to the cameras.

FIG. 5D shows filter configurations of the light sources in an MCAM system. The light sources can have filters, such as fluorescent filter or polarized filters, to provide excitation or light with specific characteristics. The light sources can have no filters in FIG. 5D(a). Some light sources can have no filters and some light sources have filters 502C in FIG. 5D(b), such as no-filtered light sources for illumination and fluorescent filtered light sources for fluorescent excitation. The light sources can have multiple types of filters 502C and 502C* in FIG. 5D(c), such as no-filtered light sources for illumination, polarized light sources for polarized light, and fluorescent filtered light sources for fluorescent excitation. Other configurations can be used, such as filtered light sources can be alternatingly arranged or randomly arranged with non-filtered or with different type filtered light sources.

3D Tracking

In some embodiments, the present invention discloses a microscope technology that offers the ability to track and image organisms or cells in large areas in 2D or 3D. The technology includes multiple cameras having overlapped fields of view, which can be utilized for depth determination using stereoscopy of photogrammetry. For example, tuning the microscope to a large amount of field of view overlap, such as at least 50% in one direction, can enable the MCAM system to perform 3D object or cell tracking and 3D organism or cell behavior analysis across a finite depth range, which is useful in certain applications of model organism behavioral or cellular study. The overlap amount can be changed by changing the magnification of the cameras or the fields of view of the cameras. For example, decreasing the magnification of each camera can increase the inter-camera overlap for the MCAM system. Alternatively, changing a lens mechanism, an optic mechanism, a sensor position in a camera, or changing a camera position can change the overlap amount.

After tuning to larger than 50% overlap, all areas of the sample are overlapped by two or more cameras. In the overlap areas, optical information about points within the specimen plane is captured by two or more cameras. Such redundant information can be used by stereoscopic and/or photogrammetry methods to obtain an estimate of object depth and/or an object depth map, which can be combined with the 2D information that is captured about object position and morphology.

With the larger than 50% overlap, all areas in the sample are captured by the cameras in two or more images. The captured images can be processed to obtain 3D positions of the objects, for example, by inputting the image data into a 3D object detection convolutional neural network (CNN), which employs stereoscopic or photogrammetry in the feature kernels.

In some embodiments, 3D object detection can be achieved by using more than one illumination patterns. Using multiple illumination patterns, the MCAM does not need to have 50% or larger overlap fields of view, even though the higher the overlap fields of view, the higher depth accuracy can be achieved. Phase difference between the light paths can be used to determine the depth of the object.

In some embodiments, 3D object detection can be achieved by using cameras in different planes. For example, a set of cameras on top of the sample can track movements of the cells in a lateral plane. Another set of cameras at a side of the sample can determine the depth information of the cells to distinguish overlapped cells showing in the lateral plane.

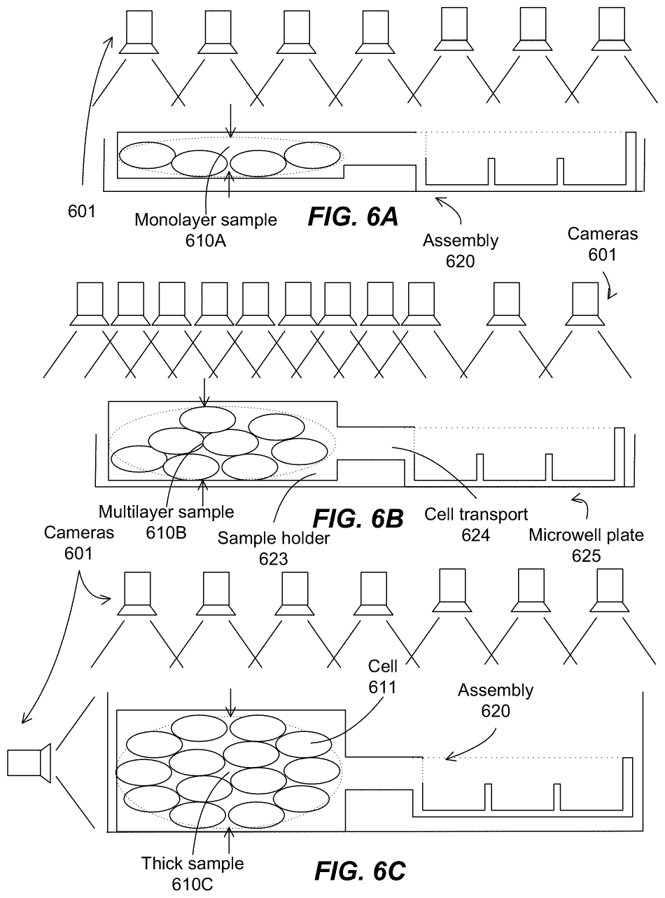

FIGS. 6A-6C illustrate configurations of an imaging system configured for 2 or 3 dimensional movement tracking according to some embodiments. An assembly fixture 620 can be configured for dissociating and transporting cells in a sample from a sample holder 623, through a cell transport section 624, to wells in a microwell plate 625. The sample holder 623 can be configured for a monolayer tissue sample 610A, multilayer tissue sample 610B, or thick tissue sample 610C. The assembly fixture can have a holder for the microwell plate, or the microwell plate can be a part of the assembly fixture. The wells in the microwell plate can be configured for a single cell, which can allow single cell analysis without cross cell data. Between the sample holder and the microwell plate is a transport section, which is configured to transport cells from the sample holder to the microwells in the microwell plate. The transport section can include multiple conduits, such as microfluidic conduits, which is configured for a single cell passage. Alternatively, the conduits can have a larger portion near the sample holder for accepting clusters or groups of cells. The conduits then can have a narrow portion, configured for mechanical sheering the cell clusters, so that only single cells pass through to the microwells. Filters can be placed in the conduits to filter out large objects, such as debris or cell clusters.

In some embodiments, the assembly fixture is at least partially transparent to the wavelengths of interest. For example, to capture images, the assembly fixture can be transparent to the visible wavelengths. To capture IR or UV images, the assembly fixture can be transparent to the IR or UV wavelengths, respectively. To capture fluorescence images, the assembly fixture can be transparent to the fluorescence excitation signals and the emitted fluorescence signals.

In some embodiments, the assembly fixture includes inputs for cell processing, such as an input for enzyme delivery to dissociate the sample into cells, an input for introducing a lysis agent to the microwell plate to remove cell membranes, or an input to introduce barcode agents to the microwell plate for identify different cells in different microwells.

In FIG. 6A, a monolayer sample 610A is disposed in an assembly fixture 620, under an array of cameras 601. After an enzyme is introduced to the assembly fixture at the sample holder section, the cells are dissociated, and then travel along the transport section to rest at the wells of the microwell plate. With a monolayer sample, the cells are dissociated in a lateral plane, and thus an array of cameras on top, at bottom, or both top and bottom can be adequate to track the movements of the cells. The cameras can have a small amount of overlap, such as less than 50% to stitch the images together to track the cell movements, such as cross cameras.

In FIG. 6B, a multilayer sample 610B is disposed in an assembly fixture 620 (note 620 is not labelled in FIG. 6B), under an array of cameras 601 to be dissociated and transported to the wells of the microwell plate. With a multilayer sample, the cells are dissociated in three dimensions, e.g., in a lateral plane with a finite depth.

In some embodiments, the cameras are configured to capture the 3D movements of the cells, for example, by having a large amount of overlap, such as more than 50% to calculate the finite depth information of the cells.

In some embodiments, the transport conduits can be configured for monolayer lateral movements. The wells can have a shallow depth to accept a monolayer of cells, such as a single cell. The cameras can be configured to have large overlap at the sample holder section, and smaller overlap at the transport and well sections.

In FIG. 6C, a thick sample 610C is disposed in an assembly fixture 620, under an array of cameras 601 to be dissociated and transported to the wells of the microwell plate. With a thick sample, the cells are dissociated in three dimensions with a large depth.

In some embodiments, the cameras are configured to capture the 3D movements of the cells, for example, by having an extra set of cameras at a side of the assembly fixture to capture depth information, in addition to the set of cameras at the top and/or at the bottom to capture lateral information. With the depth images captured by the side cameras, the controller of the microscope can be able to track the movements of the cells in three dimensions, for example, by using the depth images to distinguish cell overlap viewed in the lateral plane.

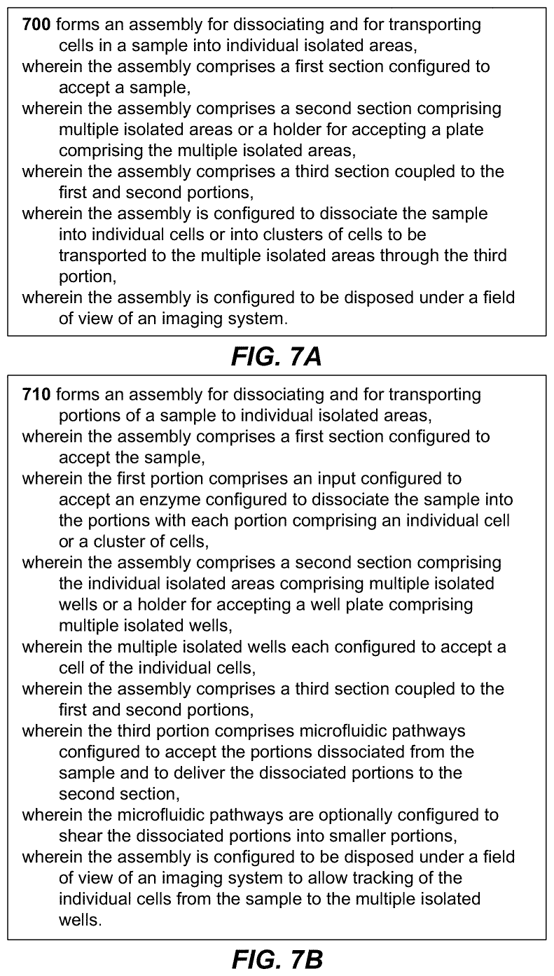

FIGS. 7A-7B illustrate flow charts for forming an assembly fixture configured for dissociating and for transporting cells in a sample into isolated areas according to some embodiments. In FIG. 7A, operation 700 forms an assembly fixture for dissociating and for transporting cells in a sample into individual isolated areas. The assembly fixture comprises a first section configured to accept a sample. The assembly fixture comprises a second section comprising multiple isolated areas or a holder for accepting a plate comprising the multiple isolated areas. The assembly fixture comprises a third section coupled to the first and second portions. The assembly fixture is configured to dissociate the sample into individual cells or into clusters of cells to be transported to the multiple isolated areas through the third portion. The assembly fixture is configured to be disposed under a field of view of an imaging system.

In FIG. 7B, operation 710 forms an assembly fixture for dissociating and for transporting portions of a sample to individual isolated areas. The assembly fixture comprises a first section configured to accept the sample. The first portion comprises an input configured to accept an enzyme configured to dissociate the sample into the portions with each portion comprising an individual cell or a cluster of cells. The assembly fixture comprises a second section comprising the individual isolated areas comprising multiple isolated wells or a holder for accepting a well plate comprising multiple isolated wells. The multiple isolated wells each configured to accept a cell of the individual cells. The assembly fixture comprises a third section coupled to the first and second portions. The third portion comprises microfluidic pathways configured to accept the portions dissociated from the sample and to deliver the dissociated portions to the second section. The microfluidic pathways are optionally configured to shear the dissociated portions into smaller portions. The assembly fixture is configured to be disposed under a field of view of an imaging system to allow tracking of the individual cells from the sample to the multiple isolated wells.

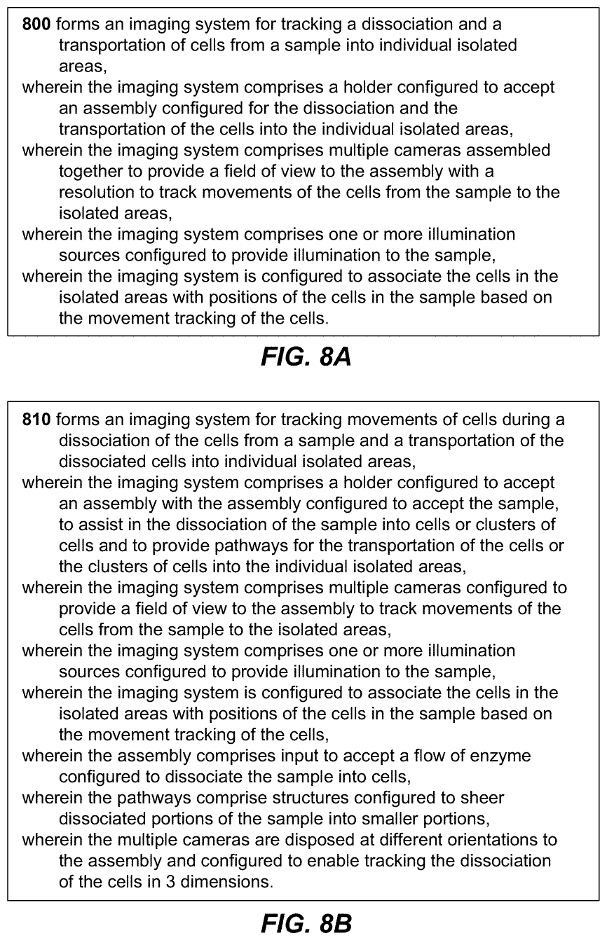

FIGS. 8A-8B illustrate flow charts for forming an imaging system configured for tracking movements of cells from a sample into isolated areas according to some embodiments. In FIG. 8A, operation 800 forms an imaging system for tracking a dissociation and a transportation of cells from a sample into individual isolated areas. The imaging system comprises a holder configured to accept an assembly fixture configured for the dissociation and the transportation of the cells into the individual isolated areas. The imaging system comprises multiple cameras assembled together to provide a field of view to the assembly fixture with a resolution to track movements of the cells from the sample to the isolated areas. The imaging system comprises one or more illumination sources configured to provide illumination to the sample. The imaging system is configured to associate the cells in the isolated areas with positions of the cells in the sample based on the movement tracking of the cells.

In FIG. 8B, operation 810 forms an imaging system for tracking movements of cells during a dissociation of the cells from a sample and a transportation of the dissociated cells into individual isolated areas. The imaging system comprises a holder configured to accept an assembly fixture with the assembly fixture configured to accept the sample, to assist in the dissociation of the sample into cells or clusters of cells and to provide pathways for the transportation of the cells or the clusters of cells into the individual isolated areas. The imaging system comprises multiple cameras configured to provide a field of view to the assembly fixture to track movements of the cells from the sample to the isolated areas. The imaging system comprises one or more illumination sources configured to provide illumination to the sample. The imaging system is configured to associate the cells in the isolated areas with positions of the cells in the sample based on the movement tracking of the cells.

The assembly fixture comprises input to accept a flow of enzyme configured to dissociate the sample into cells. The pathways comprise structures configured to sheer dissociated portions of the sample into smaller portions. The multiple cameras are disposed at different orientations to the assembly fixture and configured to enable tracking the dissociation of the cells in 3 dimensions.

Analysis at Wells

In some embodiments, a spatial biology process can be performed using an imaging system having multiple cameras for a large field of view with sufficient resolution for tracking movements of cells in a tissue sample into isolated areas such as individual microwells in a well plate. By determining the beginning and the end of each cell movements, the imaging system can associate the microwell locations to the original cell positions in the sample. As such, by analyzing the cells in the microwells, the analysis can achieve the spatial information needed for constructing a map of the analysis results relative to the cell positions in the sample.

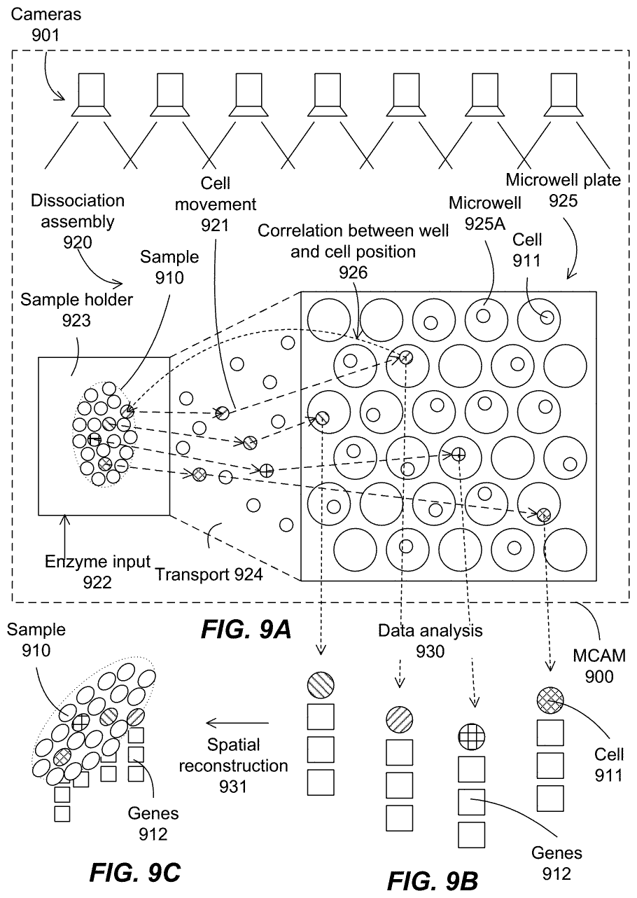

FIGS. 9A-9C illustrate a process for a spatial construction of molecular or cellular information of cells in a sample according to some embodiments. An assembly fixture 920 (Note: labelled Dissociation Assembly) is disposed in a microscope having multiple cameras 901. The microscope can be configured to have a combined field of view large enough to cover the assembly fixture, or at least cover a portion of the assembly fixture to allow tracking of cells from a sample to multiple isolated areas of microwells.

The assembly fixture can include a sample holder section 923, which is configured to accept a sample 910. The sample holder section can be configured to accept an enzyme input 922, with the enzyme configured to dissociate the sample into multiple cells 911.

The assembly fixture can further include a transport section 924, which can include multiple conduits such as microfluidic conduits configured to accept the cells dissociated from the sample holder section. The microfluidic conduits are configured to guide the cells to the microwells 925A in a microwell plate section 925.

The MCAM system 900 can be configured to sequentially capture images of the assembly fixture, e.g., the positions of the cells in the sample and the positions of the cells when the cells move along the microfluidic conduits to the individual wells of the microwell plate. Object recognition can be applied to the image sequence to track the movements of the individual cells, such as providing a cell movement 921 for each single cell from the original position in the sample to the final position at a well of the microwell plate. A correlation 926 can be formed between the original cell position in the sample and the final cell position in the well. The correlation can enable a construction of a map showing the spatial characteristics of the sample.

For example, each cell in a well can be analyzed, such as to run an analysis 930 to obtain molecular information such as gene data 912. Using the correlation between the well location and the cell position, a spatial reconstruction can be made to show a map of the sample with molecular or cellular information for each cell in the sample.

Assembly Fixture Formation

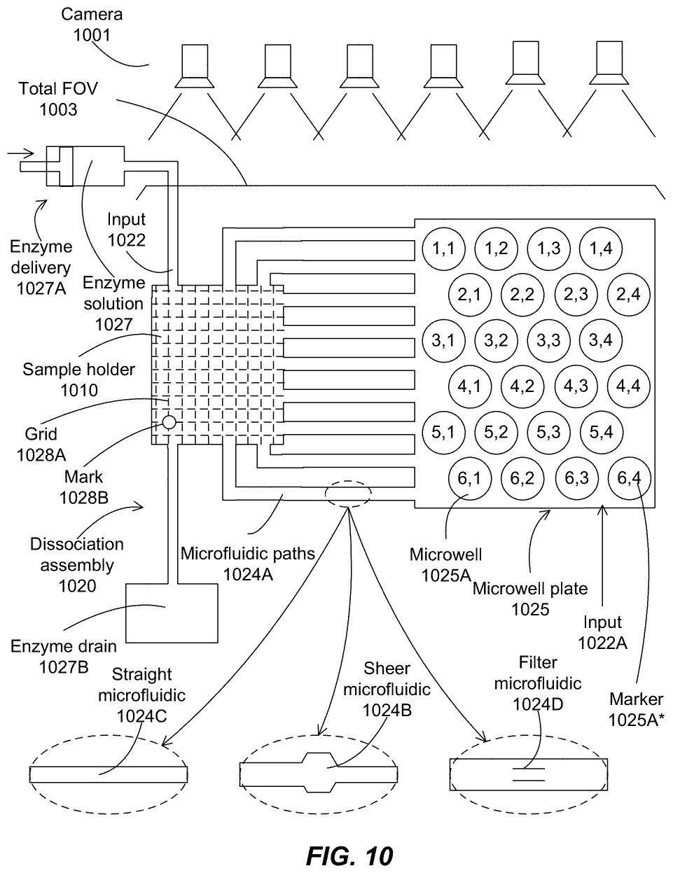

FIG. 10 illustrates a configuration of an assembly fixture for determining movements of cells according to some embodiments. In some embodiments, an assembly fixture 1020 can be used to hold the sample and to facilitate the movements of the cells dissociated from the sample into individual isolated areas of microwells. For example, an assembly fixture can have an integrated sample holder 1010 and an integrated microwell plate 1025, together with microfluidic conduits 1024A connecting the sample holder with the microwell plate. Alternatively, an assembly fixture can include a first section for supporting a sample holder 1010 and a second section supporting a microwell array plate 1025. For example, the fixture can be configured to accept a microwell array fabricated on a 1 mm thick substrate, and provide gasket support to confine the assay reagents to the microwell array. The microwell arrays are used to entrap cells, such as single cells, and beads, such as one bead per cell, within wells having small reaction chambers. The assembly fixture can also have multiple transport paths, such as microfluidic paths 1024A (including straight microfluidic conduits 1024C, sheer microfluidic conduits 1024B, or filtered microfluidic conduits 1024D), which are configured to couple the sample in the sample holder to the microwells of the microwell plate. The sample holder 1010 can include a grid 1028A or fiducial marks 1028B, which can assist in indentify position of cells in the sample. The assembly fixture, the sample holder, or the microwell array can be consumable components or reusable.

The microwell array can have surface features such as domes or ridges between the microwells 1025A that are designed to help guide cells and beads into the wells and/or prevent them from settling on the surfaces between wells. The microwell array can have markers associated with one or more microwells to provide labels 1025A* for each of the microwells 1025A.

In some embodiments, the microwell array can be configured for sealing the openings of microwells, for example, using a cap, during cell lysis steps, to prevent cross hybridization of target nucleic acid between adjacent microwells.

In some embodiments, the assembly fixture can be configured for use in optical, fluorescence imaging, or spectroscopic assessment of the sample holder, the transport section, and the microwell array section. For example, the assembly fixture can be configured to be mounted to a microscope having multiple cameras 1001 having a combined field of view 1003 that covers the entire assembly fixture, or at least the areas of the assembly fixture that enable the movement tracking of the cells during a dissociation from the sample and transportation to the microwells. The assembly fixture can be transparent to the wavelengths of interest, such as visible, IR, UV, or fluorescence wavelengths, such as fabricated from materials suitable for the spectral requirements for the imaging or spectroscopic technique used by the microscope. Alternatively, the assembly fixture can include optically transparent windows at suitable locations without obstruction for tracking cells during the dissociation and the transportation to the microwells.

In some embodiments, an assembly fixture can be configured to facilitate the pipetting or dispensing of cell enzyme and assay reagents 1027 into the sample holder section and into the microwell array section, such as having one or more inlet ports 1022 and 1022A and/or outlet ports 1027B for creating fluid connections with delivery systems for introducing reagents. The assembly fixture can include bypass channels, for example, to avoid overfilling and/or back flow of solution and reagents. In some embodiments, the assembly fixture can include mechanical sheer components 1024B and physical and/or chemical filters 1024D to prevent passage of non-selected objects, such as large molecules or debris to minimize cross-contamination in the microwells.

In some embodiments, the assembly fixture can include a pump 1027A or other fluid actuation mechanisms for control of fluid flow through the assembly fixture, such as to control a flow of enzyme to the sample holder for dissociating the sample. The assembly fixture can include drains, such as a drain 1027B for the enzyme solution, or a drain for the lysis solution. The assembly fixture can include vents for providing escape paths for trapped air. The assembly fixture can include waste reservoirs.

In some embodiments, the assembly fixture can include alignment features for easily removable and highly precise and repeatable positioning of the assembly fixture to the microscope.

In some embodiments, the assembly fixture can include temperature control components or thermal interface features for mating to external temperature control modules. The temperature control feature can be configured to provide a suitable environment for the cells to keep the cells alive during the dissociation and the transportation to the microwells for further downstream analysis.

In some embodiments, the assembly fixture can be configured to be interface with downstream analysis equipment, such as thermal cyclers for the amplification of RNA and DNA by a polymerase chain reaction (PCR), or sequencing instruments. For example, the assembly fixture can include removable collection chambers to be used in external instruments.

Setting Image Resolution

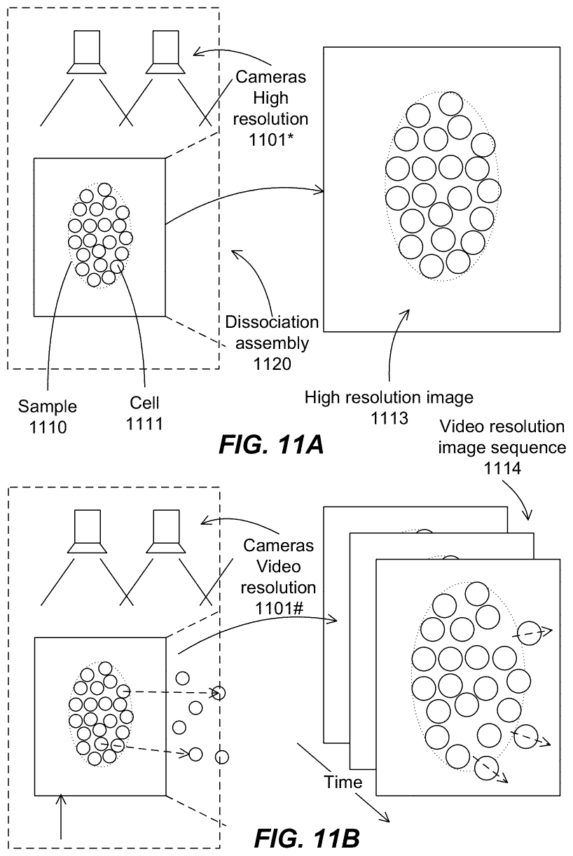

FIGS. 11A-11B illustrate a set up configuration for determining original positions of cells in a sample according to some embodiments. In FIG. 11A, the sample can be imaged at a resolution that can allow clearly distinguishing the cells 1111 in the sample. For example, the sample 1110 can be disposed in a sample holder of an assembly fixture 1120, which is then disposed in a microscope. The microscope can include multiple cameras, setting a high resolution, such as a highest resolution 1101* of the cameras. One or more high resolution images 1113 of the sample can be captured from the sample.

In some embodiments, images of the sample can be captured by a separate camera system, for example, when the resolution of the microscope is not adequate.

In FIG. 11B, the sample can be imaged at a resolution that can allow tracking of the movements of the cells 1111. For example, after the sample images are captured by the microscope at a high resolution, the microscope can be set at a lower resolution 1101# to allow fast capture of sample image sequence 1114, which can allow tracking of the cell movements based on an object recognition algorithm. The lower resolution is configured for the capture speed, which can be based on the diffusion and migration speed of the cells in the assembly fixture.

In some embodiments, the sample can be imaged with a bright field imaging process, or can be imaged with a fluorescence microscopic process, in conjunction with fluorescence stain technique. In some embodiments, multiple cameras at different angles, such as top, bottom and side, can be used for monitoring the cell movements.

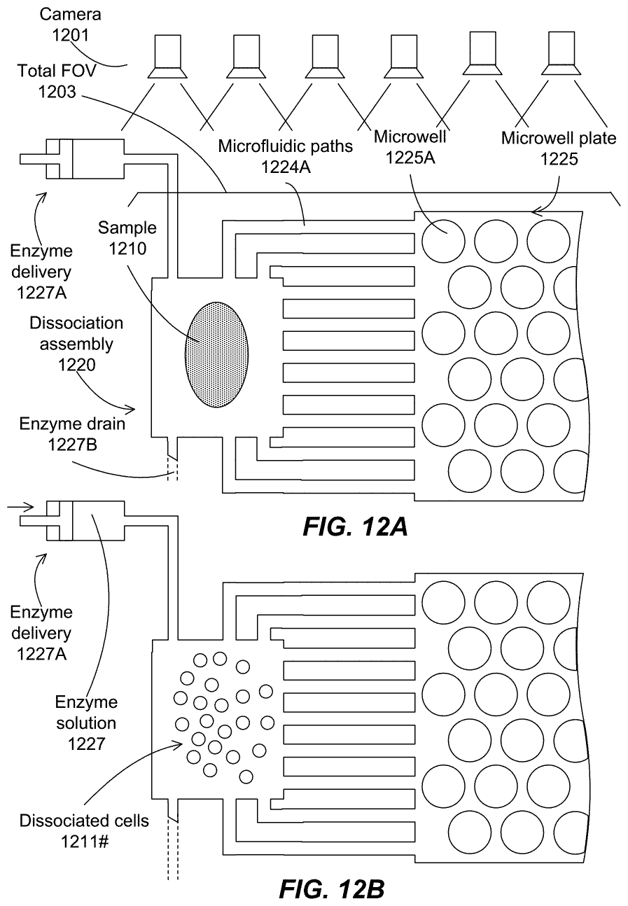

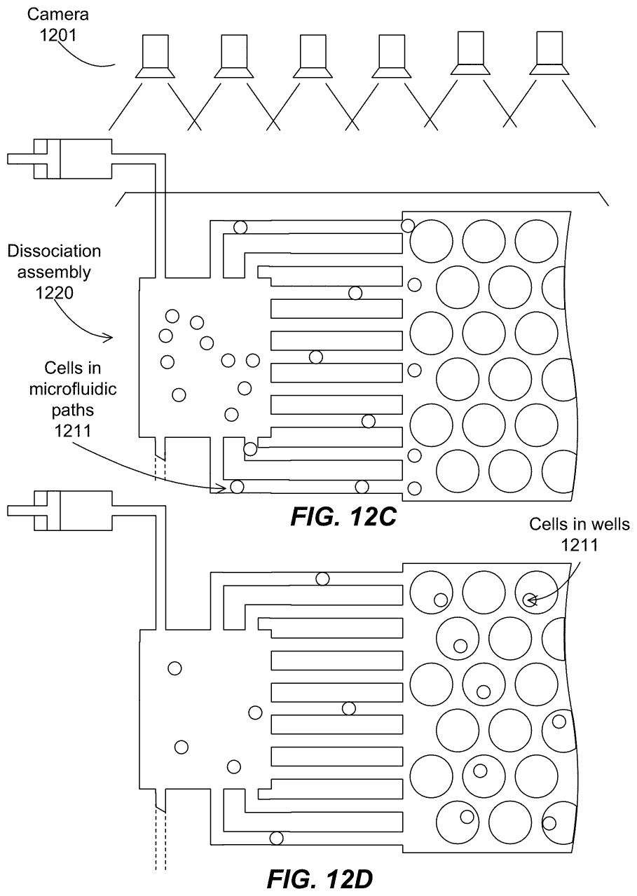

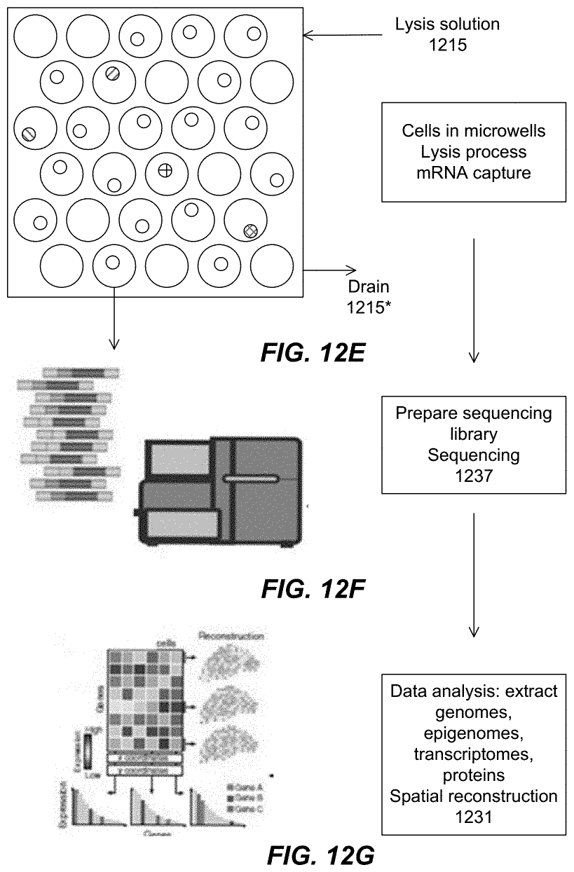

FIGS. 12A-12G illustrate a process flow for forming a spatial map of a sample according to some embodiments. The process can include a sample preparation (FIG. 12A), initial sample imaging with high resolution (FIG. 12A), cell singulation or dissociation such as with enzyme (FIG. 12B), cell movements to wells of a well plate (FIGS. 12C and 12D), cell tracking from the sample to the wells, cell analysis (FIGS. 12E and 12F), spatial assignment of cells and corresponded cell molecular or cellular information in wells to cell positions in sample (FIG. 12G).

In some embodiments, a spatial biology process can begin with an intact tissue sample, which can be immunostained, imaged, and placed into a consumable for dissociation and isolation of individual cells into microwells. Throughout the process of dissociation and single-cell isolation, a micro-camera array microscope (MCAM) is used to continuously image and track the cells. Imaging continues while assays are conducted on the live single cells in the microwells to observe their responses to stimuli. From there, the cells can be lysed, their RNA reverse transcribed into cDNA, and their cDNA ultimately sequenced by an external next-generation sequencing instrument. Finally, software is used to reconstruct the tissue section, tying the genetic expression data back to its spatial location within the tissue sample.

FIG. 12A shows an assembly fixture that maintains the tissue sample, its cellular components and cellular-sized objects (e.g., barcoded beads) in a monolayer within the field-of-view such that all of the objects are within the depth-of-field of the system and may be observed continuously across time and space throughout various analyses. The assembly fixture can include a microfluidic device that is used to deliver enzymes and shear forces to the tissue sample to break it down into individual cells.

FIG. 12B shows the tissue sample starting to break down into individual cells and other components. The microfluidic device can have outlets to recycle fluids and filter out dead cells and other debris not needed for analysis.

FIG. 12C shows the process for how enzymes break the tissue section into individual cells and the combination of shear forces and microfluidic channels flows those cells into individual wells of the well plate. The MCAM continues to track each cell through this process.

FIG. 12D shows an individual cell successfully isolated in a well. The MCAM remains in place for consistent imaging throughout the live assays and preparation for sequencing.

FIG. 12E shows an up-close view of a well-plate having a cell in a well. FIG. 12F shows a process for a preparation of a sequence library and the sequencing process. FIG. 12G shows a process for analysis, extract genomes, epigenomes, transcriptomes, proteins, and then forming a spatial reconstruction of the sample.

Sample Preparation

A microtome can be used to slice a piece of tissue removed from an organism into a thin section or other techniques such as a touch preparation (or touch imprint) can be used to transfer cells/cellular material from a tissue sample 1210 onto a sample holder such as a glass slide or other consumable. The sample holder can then be mounted to an assembly fixture 1220 having a transport section 1224A leading to a microwell plate 1225 having multiple wells 1225A. Alternatively, the sample holder is a part of the assembly fixture, e.g., the sample is disposed to a sample holder section of the assembly fixture. This may be one or several layers of cells in thickness (several microns to tens of microns thick) and an aerial size of one, two, three, four or more square centimeters in the preferred embodiment; although larger sizes such as 6 cm by 8 cm are feasible if necessary. Operating temperatures and times will be optimized to maximize cell viability and/or molecular integrity for downstream assays and workflows.

Specimen Imaging

Once the specimen is mounted onto the holder in an assembly fixture, the specimen may be imaged. Initial imaging may be accomplished with any desired microscope. While bright field microscopy is commonly used, fluorescence microscopy may also be used in conjunction with common staining practices (for example, immunohistochemistry) to obtain additional information about the sample/tissue architecture and identify molecular markers that may be indicative of disease. In some embodiments, the imaging is accomplished with the same microscope that is used for the cell tracking process. If a microscope other than the MCAM is used for the initial imaging, then a fiducial system may be used to correlate cell location in the MCAM's initial image with the image taken by another microscope. This may be done to obtain an initial image that has resolution higher than what is possible with the MCAM. The specimen is then imaged with the MCAM for continuous observation once cell singulation begins and cells can start to move from their original location in the specimen. Imaging remains continuous at video rates to track cell movement until the cells have been relocated to their final position in isolated wells in the well plate.

Throughout the process, including dissociation and cell movements, the cells are maintained within the depth of field of the MCAM imaging system such that they are continuously monitored. The depth of focus of the system depends on the numerical aperture of the optical configuration, however for the operating resolutions envisioned needed to resolve individual cells, the depth of field is sufficiently large to allow for flow and movement of cells in the assembly fixture.

Cell Singulation

In order to obtain single cells, the specimen is singulated or dissociated (FIG. 12A). Singulation may consist of many processes that can separate the specimen into single cells or smaller groups of cells. Typically, singulation can be done using a physical cutting of the specimen, laser cutting, enzymatic dissociation, vibration/sonication, other methods, or a combination of multiple methods. In some embodiments, the singulation process can be performed to singulate the specimen to parts that contain more than a single cell, even groups of cells, or it may be desirable to break down the specimen into components of cells such as the nuclei. In some cases, the entire specimen may not need to be singulated—just portions of the specimen will be singulated. Using a laser dissection tool, single cells, grids of cells or clumps of cells may be cut from the specimen.

A touch preparation is an approach commonly used by pathologists to deliver a rapid onsite evaluation and often, a preliminary diagnosis, of a tissue specimen during surgical procedures. In this approach, the tissue section is removed from the body and pressed gently against a glass slide for a few seconds, transferring cells to the slide in the process. The sample may then be stained to highlight the tissue morphology and relevant biomarkers or may remain unstained and can be used for additional analysis after the surgery. The result of a touch preparation is generally a thin layer of whole cells on a slide, often including cells of particular interest such as tumor cells. Thus, the touch preparation can be used for the gentle dissociation and continuous imaging components of this invention.

In some embodiments, the sample is gently singulated using one or more enzymes. Enzymatic dissociation of cells from their matrix components is well known. In addition, the use of a microfluidic holding device to facilitate the use of enzymes to dissociate the specimen into cells and the other specimen constituents is known.

An enzyme delivery 1227A can be coupled to the assembly fixture 1220 to deliver an enzyme to the sample. The enzyme can be drained out at an enzyme drain 1227B. The enzyme delivery can be controlled automatically by sensors or manually by an operator. The enzyme can include ligases, reverse transcriptases, polymerases, or restriction nucleases. Other reagents can be used.

Once singulated, the cells 1211 are available to be moved to the well plate by a flow fluid (FIG. 12B). In some embodiments, channels are provided to direct and control the flow and motion of the cells. For example, two different microfluidic plates can be used to enzymatically dissociate live cells from a specimen and then move the live cells in order to separate the cells and sort the cells into holding containers. All of this is accomplished in an assembly fixture, which is a single integrated microfluidic device configured to dissociate tissue samples into individual cells using continuous imaging throughout the process. Uniform illumination and continuous observation over all aspects of the microfluidic device can be provided by a microscope. In some embodiments, the cells/particles/cellular components can be retained within the depth of field of the imaging system for continuous tracking across space and time.

Cell Movement to the Well Plate

Once the cells are singulated, they are moved to a different location, for example, to wells of a well plate (FIGS. 12C and 12D). There is no requirement for any particular orderly layout of the wells. Well location and spacing may be random or organized. Well plates typically have wells in rows and columns, but this invention includes well plates with wells of any orientation, random or organized. As noted above, there are well-established techniques using microfluidic devices to conduct and move the cells from the specimen holder into wells in the well plate.

In addition to the capabilities of imaging and tracking cells, components of cells, and particles, other downstream applications can be incorporated to expand upon the system's functionality. Essentially, cells can be organized, inspected and manipulated while being able to relate their positioning and characteristics to a different formation that they held at an earlier point in time. The specimen holder or other components of the system may include any of a number of functions on the cells such as cell filtering (to isolate a certain size of cell or to remove dead cells or other unwanted material), counting, sorting, staining, electric measurements, chemistry, etc. Wells within well plates will be very valuable for isolating individual cells or groups of cells for any number of analyses ranging from culture to drug screening and molecular studies. In addition, flow cytometers are traditionally used to identify cells in fluid and this valuable technology could be incorporated. The present method and system can include two- or three-dimensional imaging of particles in a fluid to create a device that permits improved sorting of cells, counting of cells, gathering fluorescent data from cells and permits the spatial information of a specimen to be retained after such assays are performed. Any of these functions may or may not be included into a final system such that the complexity of the system can be increased to serve multiple purposes involved in research or clinical applications.

In some embodiments, a specimen may be trimmed to a nominal size of 10 mm by 10 mm. The specimen may be 20 microns thick. The open face of length 10 mm may contain several thousand cells in a strip that is 10 microns wide. At a flow rate of flow fluid that is 1 microliter per second, a shear force sufficient to dislodge cells from the cell mass is created and cells are moved toward the flow channels in a more or less continuous flow of several hundreds to thousand cells from the face of the specimen to the flow channel. To move all the cells in a 10 mm long specimen, it may take more than 16 minutes. The cross section area of the flow channels is on the order of 20,000 microns by 50 microns or 1,000,000 square microns and the flow rate is adjusted to move each cell at an average speed of 200 microns per second. With partially overlapping images of each 10 micron wide cells' position, this may require roughly a 30 frame per second frame rate on the microscope. These dimensions and flow speed create a volumetric flow of 200,000,000 cubic microns per second or 0.2 cubic mm per second (0.2 microliters per second). The entire flow liquid required is on the order of magnitude of 150 cubic mm or 0.15 cc. All of these numbers are provided as general indicators of size, speed, volumes, etc. Since cell sizes (and the sizes components of cellular matter) vary depending on the specimen, the figures here will change in each case. These figures may be different by one, two or more orders of magnitude in any specific situation.

The flow channel may be the same width as the specimen, narrower than the width of the specimen or wider than the width of the specimen. Separate mini channels may be incorporated in the flow channel to create directed flow paths. There may be 10, 100, 1000 or more distinct mini channels incorporated in the flow channel.

Cell Tracking to the Well Plate

The MCAM is used to image the specimen and capture image data of each cell as it is singulated from the specimen and then moved into wells in the well plate. This process may be happening for hundreds, thousands, or hundred thousands cells and/or particles simultaneously within the field-of-view of the MCAM. For example, a specimen of 10 mm by 10 mm may contain 1,000,000 cells in a single layer, assuming an average cell size of 10 microns. Since specimens may contain several layers of cells thus implying the cells to be moved and tracked are on the order of several million cells.

In accordance with Poisson Loading statistics, the number of wells available for isolating individual cells/particles shall be significantly more than the number of cells, for example 10-times more wells than cells. Furthermore, in some embodiments, the wells must be significantly larger than the cell itself to allow for space for both the cell and the barcoded bead. Together, the relatively large well size and large number of wells compared to the number of particles necessitates the need for the well-array area to be an order of magnitude larger than the initial specimen sample area. The large overall area defines the required field-of-view of the system.

During this transit event, each cell in motion is continuously imaged and its position tracked to ensure that the cell that departs the specimen is correctly identified as the same cell in a determined well location on the well plate. Achieving this requires adequate spatial and temporal resolution over the field-of-view. The imaging system requires a nominal throughput on the order of at least 5-10 Gigapixels per second.

The MCAM is a scalable gigapixel imaging architecture that meets the field-of-view, spatial resolution, and temporal resolution requirements identified above. Existing MCAM products achieve image capture rates >5 Gigapixels/second and demonstrate the ability to meet the application demands outlined above.

Fiducial marks may be incorporated into the specimen holder and the well plate to provide orientation to locations on each component to provide context for particle tracking algorithms. A variety of object and/or particle tracking algorithms exists and is proven to maintain unique object identity. Some have been applied to cell movement.

Cells in the microwells may be lysed. Lysis can be performed by mechanical lysis, heat lysis, optical lysis, and/or chemical lysis. A lysis solution 1215 can be provided to the microwell plate, and excess lysis solution can be drained 1215*. The chemical lysis can include the use of digestive enzymes such as proteinase K, pepsin, and trypsin. Lysis may be performed by the addition of a lysis buffer to the microwells. Lysis may be performed at a temperature of about between 4 and 30 C, for about 10 or more minutes. Other reagents can be added to the microwells, such as PCR reagents, ligation reagents, reverse transcription reagents, enzyme reagents, hybridization reagents, sample preparation reagents, and reagents for nucleic acid purification and/or isolation.

Data Analysis

After the cells have reached the wells, e.g., the cells from the sample have been isolated into single cells from a cell population of a sample, the quality of the cell isolation is evaluated, and the viability of the cells is assessed by imaging. RNA integrity can also be evaluated, for example, for single cell RNA sequencing analysis. The isolated cells are then lysed, for example, at the well plate. Afterward, an analysis can be performed, either on the wells or in external instruments. The analysis can include extraction, processing and amplification of the genetic material of each isolated cell. preparation of a sequencing library including the genetic material of the isolated cell, and then sequencing of the library, for example, by using a next-generation sequencer. For example, the genetic material of interest (DNA or RNA) is isolated and amplified to provide enough for subsequent detection as single cells typically yield only tiny quantities of DNA or RNA. The resulting material of these steps is single-stranded DNA. The amplified DNA is made into a sequencing library before being sequenced. A sequencing library is a collection of single-stranded DNA fragments derived from one cell. The sequencing process can measure different types of genetic material, such as the genome (DNA sequencing), the DNA-methylome or the transcriptome (RNA sequencing) of each cell of a population. By determining the genome of single cells, for example, the single cell sequencing process can allow the genomic heterogeneity of a cellular population to be investigated.

Spatial Data Assignment to Cell Data

The movement tracking of the cells from their positions within the sample to their discrete locations in wells in the microwell plate can provide spatial information that connects the molecular or cellular data of a single cell to the cells' original location in a specimen and to provide this spatial data for each and every cell in the specimen. The invention uses a microscope to image the cells in the specimen and then to track each cell as it is singulated from the specimen and then moved to a particular well in a well plate. A Cartesian coordinate gridding can be used to establish the position of specific cells/groups of cells/cellular components on the specimen and their position within wells in the well plates. The grid may be set at an arbitrary fineness. The microscope and object tracking software track the cells as they move to the well plate and into wells. Assays can be run on the cells—small molecule screening, metabolic assessment, growth, etc. This information can be saved on a per-cell basis since each cell is distinguishable and isolated in individual wells. The aforementioned barcoding approach can then be used such that each of these wells has a bead with a unique barcode that is associated with its position on the well plate. Molecular contents of each cell may be attached to oligonucleotides on the bead, each oligo having the same positional barcode element that identifies it with a particular well. All of the oligos on all of the beads may be mixed together and sequenced on an Illumina sequencer, for example. And since each oligo has a location-identifying barcode, the molecular data for the oligos may be attached to the well location and the well location is likewise attached to the original cell location. A database can be used to track each cell's identity, well location, and location in the original specimen. Thus, spatial information as to the cells' original location in the specimen can be known for each cell and its associated molecular data which are tied together via the unique barcode for the location in the well plate.

Visualization of Data

Part of the invention is to provide a useful tool for visualizing the data. Software programs currently exist for spatial visualization, so those can be integrated into this system or unique software can be developed.

Instrument Systems

In some embodiments, the assembly fixture can incorporate microwell arrays integrated with flow cells coupled to a sample holder, together with necessary instrumentation to provide control and analysis functionality such as fluidics control, temperature control, external inputs and outputs such as bead distribution and collection mechanisms, and cell lysis mechanisms. For example, the temperature control can be configured for providing viability to the cells and for facilitating the accuracy and reproducibility of assay results. The assembly fixture can be disposed in a microscope, which can be integrated with the assembly fixture to provide imaging capability and image processing.

Data Analytics

The invention inherently captures very large amounts of data and can be used to further increase the data that is extracted in the process. While the spatial data and molecular or cellular data sets can be joined, the invention can be used to capture rich information on cell dissociation, cell viability, cell counting and cell statistics, to name a few. All of this data can be integrated into the data set and machine learning techniques can be applied to tease out additional insights for researchers.

System Processor and Software

In some embodiments, the spatial biology system can include a processor in a microscope or an external data processing system such as a computer, together with software to provide instrument control functionality, image processing and analysis capability, and data storage, analysis, and display functionality.

In some embodiments, the spatial biology system requires the control of several subsystems and the calculation of spatial relationship data for each cell. A central computer may be used, however, those skilled in the art will recognize that the actual physical hardware for the central computer may be a distributed group of computers, a number of sections of many computers for shared memory and compute power or even “cloud computing” where memory and computing resources are provided remotely and as needed for the computations. A software program can be used to program the computer's activities. In another embodiment, individual controllers for each subsystem are provided and these may be software, hardware or a combination of both and may reside in the central computer. The user may define a variety of inputs to the control software. For example, the user may only want to track cells of a certain size or type. Or, the user may define the maximum time limit for the entire operation. Or, the user may define how much enzyme they want circulated into the specimen holder and flow rates and changes to flow rates for the enzyme injection. These are just examples and in no way limit the types of user defined inputs that may be entered into the control system. These and many other inputs can be made by the user to optimize their particular operation. The MCAM has many settings which may be optimized depending on the specimen and controlled by the MCAM controller. For example, some settings may include illumination, focus, type of microscopy such as bright field or fluorescence microscopy, to name a few. In one embodiment, the well plate location relative to the specimen holder is determined by a stage that is controlled by a stage controller. This may be a one axis stage or a multi-axis stage depending on the type of set up, the type of specimen holder and the type of well plate being used in the process. In some cases the input of one controller can be used to determine some of the controls of another controller. For example, image data from the MCAM can provide input for the well plate stage controller to increase or decrease its speed of movement or to change directions, for example, when the last well in a set of well plate rows is filled. Or the MCAM data may be used by a fluid controller to increase the flow rate of flow fluid from one or more pumps in order to spread out or compress the distance between cells in the flow channel. A specimen holder controller can be used to maneuver the stage with the specimen as needed. The central computer provides a data stream to a storage device to retain images and data from the process. The spatial data can be computed by the central computer.

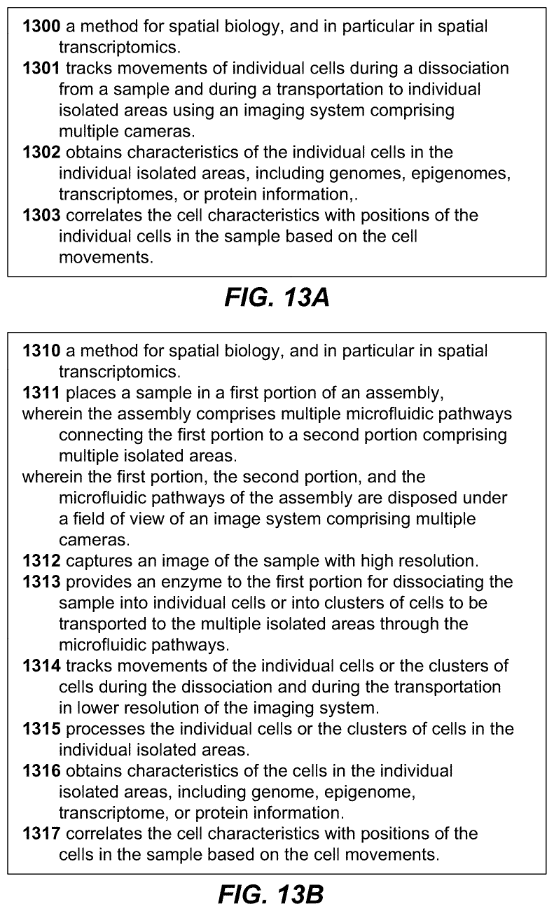

FIGS. 13A-13B illustrate flow charts for a spatial construction of molecular or cellular information of cells in a sample according to some embodiments. In FIG. 13A, operation 1300 discloses a method for spatial biology, and in particular in spatial transcriptomics. Operation 1301 tracks movements of individual cells during a dissociation from a sample and during a transportation to individual isolated areas using an imaging system comprising multiple cameras. Operation 1302 obtains characteristics of the individual cells in the individual isolated areas, including genomes, epigenomes, transcriptomes, or protein information. Operation 1303 correlates the cell characteristics with positions of the individual cells in the sample based on the cell movements.

In FIG. 13B, operation 1310 discloses a method for spatial biology, and in particular in spatial transcriptomics. Operation 1311 places a sample in a first portion of an assembly fixture. The assembly fixture comprises multiple microfluidic pathways connecting the first portion to a second portion comprising multiple isolated areas. The first portion, the second portion, and the microfluidic pathways of the assembly fixture are disposed under a field of view of an image system comprising multiple cameras. Operation 1312 captures an image of the sample with high resolution.

Operation 1313 provides an enzyme to the first portion for dissociating the sample into individual cells or into clusters of cells to be transported to the multiple isolated areas through the microfluidic pathways. Operation 1314 tracks movements of the individual cells or the clusters of cells during the dissociation and during the transportation in lower resolution of the imaging system. Operation 1315 processes the individual cells or the clusters of cells in the individual isolated areas. Operation 1316 obtains characteristics of the cells in the individual isolated areas, including genome, epigenome, transcriptome, or protein information. The characteristics can include nucleic acid variants, mutants, polymorphisms, inversions, deletions, reversions and other qualitative events found in a population of RNA or DNA molecules, such as gene expression or allelic distribution. Operation 1317 correlates the cell characteristics with positions of the cells in the sample based on the cell movements.

In some embodiments, the spatial biology method can include amplifying, sequencing, determining an amount of the target nucleic acid or complement thereof. The amplifying can include reverse transcribing the target nucleic acid, or employ a method of PCR, nested PCR, quantitative PCR, real time PCR, digital PCR, and any combination thereof.

In some embodiments, the spatial biology method can include distributing one or fewer cells to each of the wells in a microwell plate. The cells can be lysed in the microwell plate. The method can further include synthesizing cDNA in the microwell plate. Synthesizing cDNA may comprise reverse transcription of mRNA.

In some embodiments, the MCAM organism tracking includes detecting the organisms of interest, e.g., cells, in the MCAM imaging area. For example, the desired outputs from the MCAM video object tracking include a set of coordinates that each defines the 2D or 3D location and bounding box encompassing the objects of interest within the MCAM field of view as a function of time. To enable rapid detection of objects within the MCAM field-of-view, it can be beneficial to store information about the features of the object types to be tracked. The object feature information can take the form of a look-up-table, list of variables with associated values, or any other type of numeric array.

The object feature information can include measurements of the objects, such as the sizes, shapes, and dimensions of the objects, which can enable the processors to distinguish a debris from the target organisms among the detected objects. As discussed above, the MCAM related information can enable actual measurements of the detected objects, and a comparison with the object measurements in the object feature information can allow the processors to accept detected objects as the target organisms and to reject detected objects not conforming to the measurements of the target organisms.

The object feature information can include detection characteristics, which can enable the processors to select optimum detection algorithm and to perform the selected algorithm. The detection characteristics can include specific features of the target organisms, e.g., the features that can enable the recognition and identification of the target organisms. For example, for frame-to-frame detection, a threshold value between change and no change can be stored. Similarly, for edge detection, a threshold value between edge and no edge can be stored. For projection detection, a threshold value between detection and no detection can be stored. Also, fitted curves for the projection detection can also be stored. For object detection convolutional neural network, the detection characteristics can include feature detection convolution filters, wavelet filters or other filters that are object or organism specific.

The object feature information can also take the form of a pre-trained neural network, such as a convolutional neural network (CNN), which is designed for object detection, object segmentation, object tracking or a related task. More than one CNN weights can be pre-set or pre-determined via supervised learning approaches, using either MCAM image data or similar image data, of examples of the desired object types to be tracked. Some weights can also be not determined, e.g., the weight values can be left un-initialized, and to be optimized at a later time after acquisition of additional image data, using new during or after image capture.

Objects or partial objects in the captured images from individual cameras can be detected, using input information related to the target organisms, such as threshold or fitting curve values for the detection algorithms, or specific features of the target organisms such as feature filters for CNN object detection.

The detection process can include an edge detection (2D or line, monochrome or color), a projection detection, or a neural network detection (2D or 3D). The detection process can be performed at the main processor, if not being performed at the pre-processors or if there are no pre-processors. The detection process can detect whole object if the object is within the field of view of the camera. The detection process can detect a partial object, e.g., a portion of the object, if the object is shared between the fields of view of multiple neighbor cameras. Outputs of the detection process can include the image segments surrounding the objects or the partial objects. Bounding boxes and locations for the objects meeting the characteristics of the input object data can be formed. The bounding boxes and locations can be used to form tracking data of the objects.

Barcode Beads

In some embodiments, the present spatial biology method can include the use of barcoding. Barcoding is a known technique to uniquely identify molecular contents from cells, for example, RNA transcripts. Barcoding the contents of individual cells can allow the assignment of a unique signature to each cell (or the RNA in a cell), which then can enable the identification of the individual cells when the cells are mixed in a pool for batch processing with next-generation sequencing. For example, the sequencing readout also provides the barcode information which can enable associating the beads with the individual cells.

A common method of barcoding is with oligo-coated beads with millions of oligos per bead. Barcoded beads can be positioned, each into a well in a well plate, such placing the beads in a solution and depositing the solution, such as by pipetting, onto the array of wells. The beads enter the wells by settling into position with gravity or with centrifugal assistance. The beads can be loaded into the wells either before or after the cells are loaded into the wells. Wells can be designed with optimal shapes, diameters and depths to promote co-isolation of a single cell and a single bead.

Each bead can include a library of oligonucleotide probes for use in labeling and digital counting of the cellular mRNA molecules in the cell. For example, each bead can carry a unique barcode to be associated with a cell. Once positioned in the wells, the barcode of each bead can be read, which can associate the barcode information with the well and the cell in the well, e.g., the barcode information can be used to determine the cell and the well containing the cell. Each oligo can carry unique molecular identifier information (UMI), which can differentiate different molecules, such as an RNA, that binds to the oligo.

The sequencing readout provides the barcode and the unique molecular identifier information, together with the molecular information of the molecule bound to the oligo. As such, the molecular information from the oligos can be associated with the individual cells. In addition, using the unique molecular information, the number of molecules can be quantified, e.g., multiple copies of RNA transcripts can be counted for each bead.

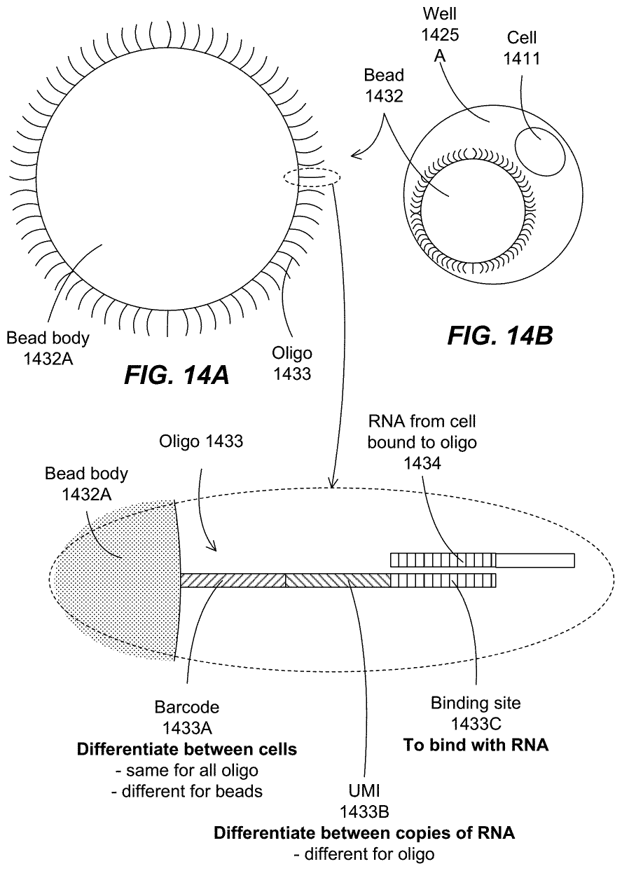

FIGS. 14A-14B illustrate a barcode bead configuration according to some embodiments. FIG. 14A shows a schematic of a bead 1432, which has a core 1432A and multiple oligos 1433 coated on a surface of the bead. In some embodiments, the oligo can have a barcode portion 1433A, which is the same for all oligos on a bead surface, e.g., the barcodes are different for different beads. Information from the barcode can be used to differentiate between beads, and since each bead is coupled to a cell, the barcode can be used to differentiate between cells.

The oligo can have a unique molecular identifier (UMI) 1433B, which are different for different oligos. Information from the UMI can be used to differentiate between copies of RNA bound to the oligos. For example, the number of UMI linked to a same RNA can be used to count the number of RNA.

The oligo can have a binding side 1433C, which, for example, is configured to bind with RNA. For example, RNA 1434 from a cell can be bound to the binding site 1433C of an oligo 1433. The sequencing process can provide the molecular information of the molecule, such as the RNA, bound to the binding site 1433C of the oligo 1433, together with the information of the barcode 1433A and the UMI 1433B. Thus, the cells from different wells in a well plate can be mixed and then sequenced together while maintain their identities due to the barcodes, e.g., the molecular information can be sorted based on the barcode information. With the barcode information linked to the well locations, the molecular information can be traced back to the cell positions in the sample to enable a construction of a sample map of the molecular information.

FIG. 14B shows well 1425A having an oligo bead 1432 and a cell 1411. The distribution of cells and beads to the microwell plate can be designed to obtain at most one cell and one bead per well. Since the well plate is still under the observation of the microscope, e.g., the well plate is within the field of view of the microscope, the cell and well distribution can be characterized to achieve a large number of wells having a single cell and a single bead. The wells without cells or beads can be eliminated, e.g., without further downstream analysis. The wells having multiple cells or multiple beads can be marked, so that the analysis can proper assess the molecular results. For example, if there are two beads in a well with a single cell, the molecular information obtained from the two beads can all be attributed to the single cell. If there is one bead in a well with two cells, the molecular information obtained from the bead can be attributed to the two cells.

In some embodiments, the spatial biology process can include preparing a sample suitable for a microscope. For example, the sample can be prepared to be about one monolayer thick, and the microscope can be set to observe a lateral plane for the movements of cells. If the sample is multilayer thick, the microscope can be set to have high overlaps between the cameras to allow for the finite depth of the sample, e.g., to track cell movements in three dimensions. If the sample is thick, the microscope can have side cameras, especially having fields of view at the sample section to track the movements of the cells. High resolution images of the sample can be captured, to obtain initial positions of the cells in the sample.

After preparing the sample, the sample can be disposed in an assembly fixture, e.g., in a sample holder section of the assembly fixture. Sample singulation or dissociation can be performed, for example, by introducing enzyme to the sample holder section of the assembly fixture. Sequences of images can be captured, with adequate resolution to distinguish between the cells and with an adequate capture speed to track the movements of the cells. The resolution of the cameras in the microscope can be adjusted from the high resolution to a lower resolution, such as the video resolution to accommodate the high speed capture.

After the cells from the sample have settle into the wells of the well plate section in the assembly fixture, for example, after the sample has completely dissociated, the well plate can be observed to characterize a distribution of the cells in the well plate.

Oligo-beads can be introduced to the wells, for example, by adding the bead solution to the well plate to let the beads settled in the wells. The process can be observed since the well plate can still be under the observation or image capturing of the microscope to characterize a distribution of the beads and cells in the well plate. Uncaptured oligonucleotide conjugated beads can be removed, for example, by washing away with a buffer solution. The wells can be covered, e.g., sealed to prevent the contents of one well diffusing into another well.

In some embodiments, the cells in the wells can be purified prior to being contacted with an oligonucleotide bead, for example, using antibodies, molecular scaffolds, beads, or by flow cytometry.

The cells in the wells can then undergo a lysis process, which can destroy cell membrane to access molecular contents of cell. For example, a lysis solution can be added to the well plate to be distributed to the wells. Once the cells are lysed, the content of the cell, such as the mRNA from the cell, can bind to the synthetic oligos at a universal binding site, e.g., oligo dT primer.

At this point, the barcode of each bead is not known, which should be determined and related to the cell that is co-isolated in the well in order to create a link between the spatial identity (start and end location) of each cell and its barcode identity (sequencing readout and molecular information). This can be done through a sequential in-situ sequencing by ligation or other methods. The MCAM can be equipped with fluorescence filters to execute the barcode determination with adequate resolution and sensitivity.

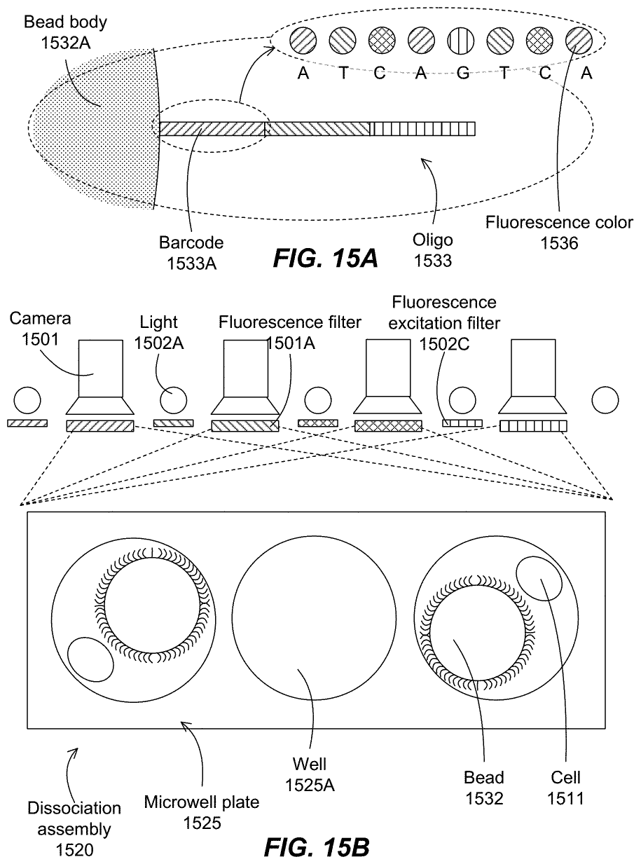

FIGS. 15A-15B illustrate a barcode characterization according to some embodiments. In some embodiments, to be used in combination with direct external barcoding or as an alternative, in-situ sequencing-by-synthesis based approaches can be used to uniquely identify cells, detect and quantify RNA transcripts, and differentiate cells based on genetic variation. An enzyme solution can be delivered to the assembly fixture, such as to the well plate, to reverse transcribe mRNA into cDNA. Padlock probes, which are fluorescent, can hybridize to the cDNA and a process of rolling circle amplification can be used to produce high levels of fluorescent signal that can be detected at a relatively low magnification level, such as 10x. These fluorescent signals can be used to decode the actual sequence of nucleotides of the mRNA and/or DNA through sequential rounds of fluorescence imaging where different signals (colors) in each round correspond to different nucleotides. The architecture is flexible and can be adjusted for specific oligo-beads. There are tradeoffs with this approach compared with the barcoded beads and next-generation sequencing approach, but can be preferred in some applications when considering the overall goal of the workflow.

FIG. 15A shows a bead 1532A with an oligo 1533. The barcode 1533A of the oligo can be determine with a sequential 4-channel fluorescence process, which can sequentially detect different colors 1536 in each step of the sequential 4-channel fluorescence process, with the different colors corresponded to different nucleotides.

The sequential fluorescence process can be performed in-situ in the microscope, for example, after dispensing the beads to the wells, before or after lysing the cells. FIG. 15B shows a configuration to perform sequential fluorescence with the microscope, with the microscope having light sources 1502A and cameras 1501 having a field of view on the assembly fixture, and particularly on the well plate 1525, with the beads 1532 and cells 1511 disposed in some of the wells 1525A of the well plate 1525.

Some of the light sources can be configured to emit fluorescence excitation signals corresponded to the nucleotides of the barcodes of the beads, e.g., in addition to the light sources configured for visible, IR, or UV radiations. In some embodiments, the light sources 1502A can be equipped with filters, such as fluorescence excitation filter 1502C to let the fluorescence excitation signals to pass through. There can be 4 different filters, and the controller of the microscope can control the light sources to sequentially turn on to generate 4 different fluorescence excitation signals in sequence.

Some of the cameras can be configured to capture fluorescence signals emitted by the barcodes, e.g., in addition to the cameras configured for capturing visible, IR, or UV radiations. In some embodiments, the cameras 1501 can be equipped with filters, such as fluorescence filter 1501A to let the fluorescence signals to pass through. There can be 4 different filters, and the controller of the microscope can control the cameras to sequentially capture images based on the different fluorescence excitation signals.

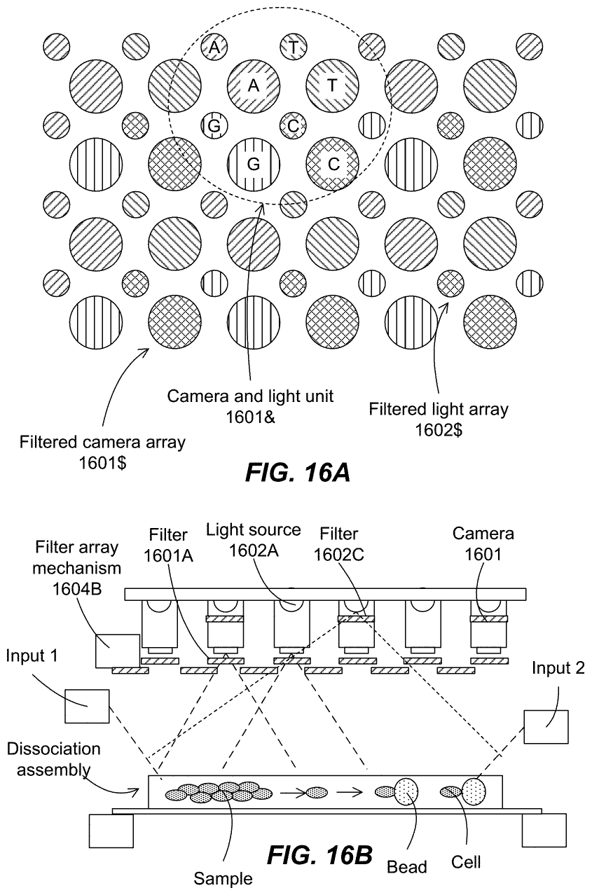

FIGS. 16A-16B illustrate filter configurations for barcode sequential fluorescence capture according to some embodiments. In some embodiments, the MCAM microscope can be configured to have fluorescence imaging capability for capturing multiple types of images, such as visible light images, and multiple types of fluorescence images for a sequential multi-channel fluorescence process. The camera array can be formed by making alternate cameras suitable for capturing different radiation, or by forming a moving filter assembly to convert the cameras to cameras suitable for different radiation.

FIG. 16A shows a configuration of a filtered camera array 1601$ and a filtered light array 1602$ for forming alternate cameras and light sources suitable for capturing 4 different radiation, such as making multiple groups of camera and light unit 1601&. Different filters can be disposed on different cameras and light sources in each group of camera and light unit 1601&. A controller can be used to control the illumination system to provide different fluorescence excitation illumination patterns to the sample.

In some embodiments, the light sources are equipped with different types of fluorescence excitation filters, in order to generate different types of radiation capable of fluorescence exciting the sample in different fluorescence modes. The different types of filters mean filters having different wavelength ranges, such as filters having different band pass ranges.

The cameras are equipped with different types of corresponding emission filters, in order to capture different types of fluorescence signals emitted by the barcodes of the beads due to the different fluorescence modes.

For example, a group of camera units can include 4 filtered camera units with different types of emission filter. Alternatively, a group of camera units can include 4 filtered cameras for barcode determination, and an unfiltered camera for cell movement tracking. The cameras can be configured to have overlap in visible light for motion tracking, and can have no overlap with only views on the wells for fluorescence determination of the barcodes.

FIG. 16B shows a configuration of a filtered light array 1602$ for forming alternate cameras and light sources suitable for capturing 4 different radiation, such as making proving filters 1602C for some light source 1602A. Different filters can be disposed on different light sources to enable the generation of filtered light sources having fluorescence excitation signals. A controller can be used to control the illumination system to provide different fluorescence excitation illumination patterns to the sample.

A filter array mechanism 1604B can be configured to provide the camera array 1601 with different filters. For example, the filter array mechanism can move to a first position to provide the cameras with a first type of filter, which can correspond to a first type of fluorescence excitation filter of the light sources. The filter array mechanism can subsequently move to different positions to provide the cameras with different types of filters for different types of fluorescence excitation signals. There can also be a position with no filters for the cameras to capture images without any filters, such as to capture visible light images. A controller can be used to control the filter array mechanism to appropriate positions corresponded to the illumination patterns generated by the light sources to match fluorescence excitation filters to fluorescence filters.

In some embodiments, the MCAM microscope can include a set of fluorescence excitation filter for the light sources and a set of corresponding emission filter for the cameras. The set of emission filters can be configured to be placed on the lenses of the cameras, to limit the radiation captured by the cameras to the wavelength range determined by the emission filter. The set of emission filters can include one or more large plates for covering multiple cameras, or can include multiple individual covers for individual cameras. The set of emission filters can be permanently coupled to the cameras. Alternatively, the set of emission filters can be movable, controlled by a mechanism to provide different emission filters to the cameras at different time, e.g., in sequence.

The set of fluorescence excitation filters can be configured to be placed on the light sources to limit the radiation generated by the light sources to the wavelength range determined by the fluorescence excitation filter. The set of fluorescence excitation filters can include one or more large plates for covering multiple light sources, or can include multiple individual covers for individual light sources. The set of fluorescence excitation filters can be permanently coupled to some of the light sources to sequentially generate different fluorescence excitation signals. In some embodiments, the set of fluorescence excitation filters can be movable, controlled by a mechanism to provide different fluorescence excitation filters to the light sources at different time, e.g., in sequence.

In some embodiments, filters having a multiple band pass property, such as dual band pass filters, can be used for the cameras. The dual band pass filters can allow a camera to capture images from two distinct ranges of frequencies without changing filters, as in the case of single band pass filters. Other multiple band pass filters can be used, such as three band pass filters or fourth band pass filters. The band passes for different ranges of frequencies can be discrete, e.g., separated by a gap between the band passes. The band passes for different ranges of frequencies can be overlapped, e.g., there is a frequency range overlapped between the band passes.

In some embodiments, the multiple band pass property of a multiple band pass filter can be characterized on the frequency ranges of interest, for example, for color ranges as in the case of fluorescence emission signals. For example, a band pass filter of 590-620 nm is configured to accept orange radiation, and a band pass filter of 620-750 nm is configured to accept red radiation. A dual band pass filter for red and orange radiation can have a frequency range of 590-750 nm, which can be considered as a single band pass filter of 590-750 nm, and can be considered as a dual band pass filter for red and orange fluorescence emission.

In some embodiments, the multiple band passes of a multiple band pass filter can be discrete. For example, a camera can be coupled to a dual band pass filter having a band pass for green color (such as 490-560 nm) and a distinct band pass for red color (620-750 nm). When a sample is excited with an excitation in the blue (450-495 nm) to ultraviolet (100-400 nm) range, the green fluorescence protein in the sample can emit a green signal (e.g., 490-560 nm) that can be captured by the camera. When a sample is excited with an excitation in the green (495-570 nm) to ultraviolet (100-400 nm) range, the red fluorescence protein in the sample can emit a red signal (e.g., 620-750 nm) that can be captured by the camera. Thus, the camera can be configured to capture fluorescence signals from two frequency ranges, without the need for a moving part, such as the need to replace the green filter with the red filter.

In some embodiments, the multiple band passes of a multiple band pass filter can be overlapped. For example, a camera can be coupled to a dual band pass filter having a band pass for green color (such as 495-560 nm) and a band pass for cyan color (450-520 nm). The band passes for these colors can be overlapped. When a sample is excited, the green fluorescence protein in the sample can emit a green signal that can be captured by the camera. When a sample is excited, the cyan fluorescence protein in the sample can emit a cyan signal that can be captured by the camera.

Alternatively, a filter can have a wide band pass of 450-560 nm, which can function as a dual band pass filter for green and cyan colors. Alternatively, a filter can have two discrete band passes of 450-510 nm and 530-560 nm for the cyan and green colors, respectively.

In some embodiments, a dual band pass filter can be designed using two dissimilar band pass filters in parallel or cascade with common input-output coupling. For example, a dual band pass filter can include a short-circuited λ/4 resonator and an improved stepped impedance resonator (SIR), using a split-ring scheme to achieve a small circuit size. Alternatively, dual band pass filter can be designed with λ/4 resonators, with two separate band passes created from one initially wide band pass in series with a stopband within the wide band pass.

In some embodiments, the multiple band pass filters can be used for the light sources, as filters for generating fluorescence excitation. The multiple band pass filters, when used with the light sources, can allow the light sources to provide more than one fluorescence excitation to the sample.

In some embodiments, using a proper selection of multiple band pass filters for excitation and emission, e.g., for the light sources and for the cameras, a microscope can capture the desired fluorescence signals, e.g., the desired colors or wavelength ranges, with other wavelengths effectively blocked out.

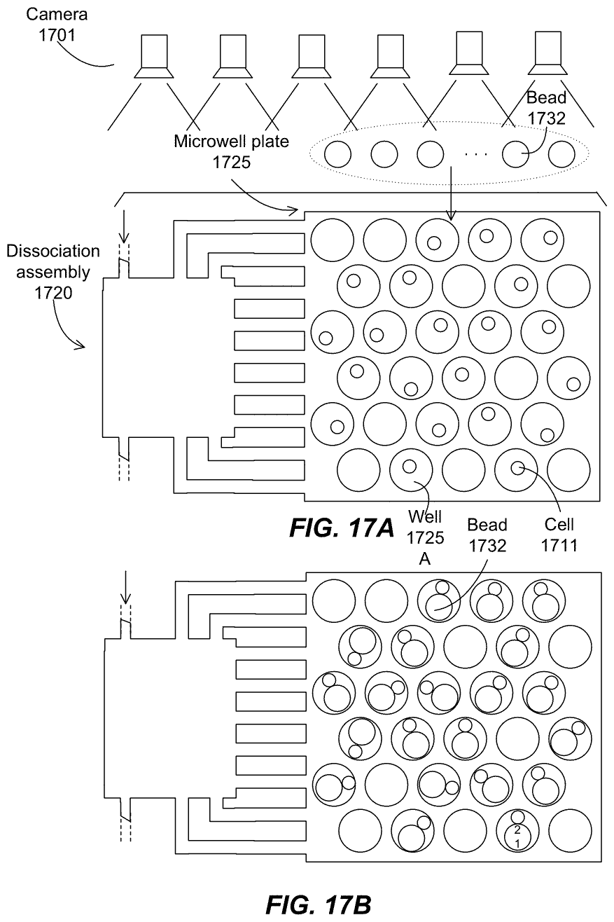

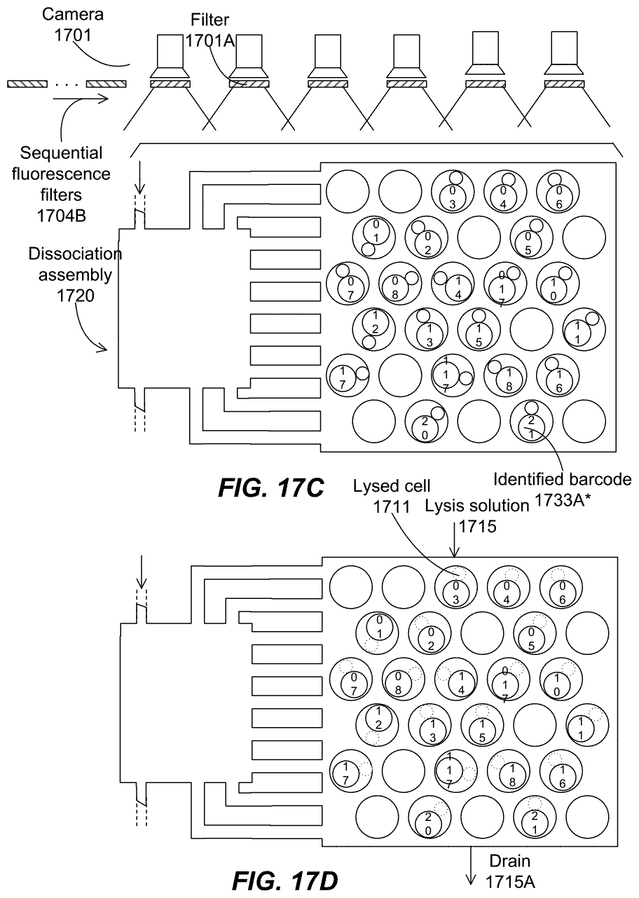

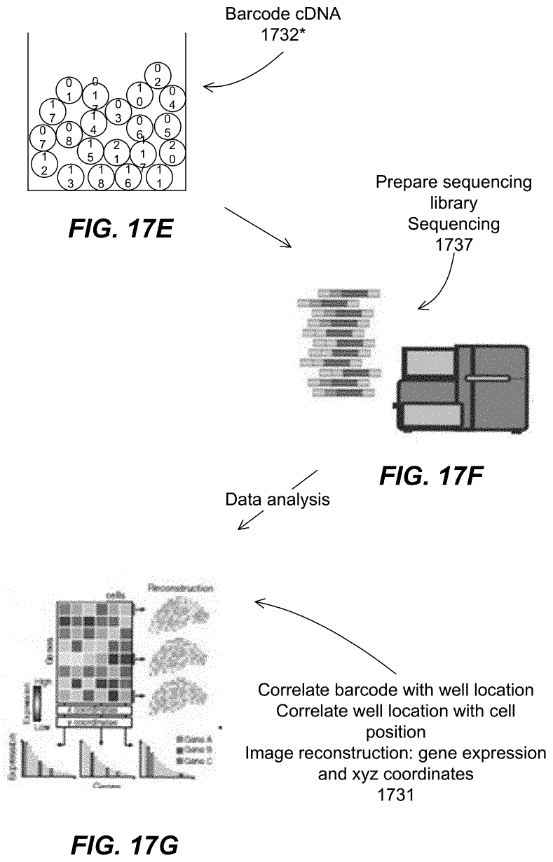

FIGS. 17A-17G illustrate a spatial biology process using barcode beads for labeling cell content according to some embodiments. The process can include a sample preparation (shown previously), initial sample imaged with high resolution (shown previously), cell singulation or dissociation such as with enzyme (shown previously), cell movements to wells of a well plate and cells at wells (shown previously), cell movement tracking from the sample to the wells, barcode beads added to wells (FIGS. 17A and 17B), barcode identification (FIG. 17C), cell lysis process (FIG. 17D), collection of cells from wells to be analyzed (FIG. 17E), cell analysis (FIG. 17F), spatial assignment of cells and corresponded cell molecular information in wells to cell positions in sample (FIG. 17G).

In some embodiments, a spatial biology process can begin with a tissue sample, which can be placed into a consumable such as an assembly fixture, for dissociation and isolation of individual cells into microwells. Throughout the process of dissociation and single-cell isolation, a micro-camera array microscope (MCAM) is used to continuously image and track the cells. Imaging continues while assays are conducted on the live single cells in the microwells to observe their responses to stimuli. Barcode beads having barcoded oligonucleotides can be added to the wells to allow sequencing the cells together. The barcode beads are then identified, such as by a sequential 4-channel fluorescence process. The cells can be lysed, then collected, their RNA reverse transcribed into cDNA, and their cDNA ultimately sequenced by an external next-generation sequencing instrument. Alternatively, the cells can be lysed before identifying the barcode beads. Finally, software is used to reconstruct the tissue section, tying the genetic expression data back to its spatial location within the tissue sample.

FIG. 17A shows an assembly fixture 1720 under the field of view of multiple cameras of a microscope. The assembly fixture can be configured to maintain the tissue sample, its cellular components and cellular-sized objects (e.g., barcoded beads) in one or more monolayers within the field-of-view such that all of the objects are within the depth-of-field of the system and may be observed continuously across time and space throughout various analyses.

After dissociating a tissue sample, the cells 1711 can travel to the wells 1725A of a well plate 1725. The individual cells are then isolated in the wells, for example, with at most one cell per well, e.g., there are well without any cell. In some cases, there can be more than one cell per well. The microscope can track each cell through the dissociation and transportation to the wells 1725A, and also identify the number of cells in each well. Beads 1732 can be added to the microwell plate 1725, for example, by using a solution containing the beads.

FIG. 17B shows the well plate 1725 having cells and beads in the wells of the well plate. The process can be observed by the microscope, for example, to identify the number of cells and beads in each well. For example, thousands or millions of cells can be isolated in the well array plate, such as a picowell plate or a microwell plate. Microfluidic conduits can facilitate the co-location of barcoded beads alongside each cell for the purpose of downstream sequencing. The MCAM remains in place for consistent imaging throughout the live assays and preparation for sequencing.

FIG. 17C shows an identification of the barcodes of the beads in the wells, using a sequential multi-channel fluorescence process. For example, a filter fluorescence mechanism can move a set of fluorescence filters 1704B in front of the cameras, so that the cameras 1701 can have an appropriate filter 1701A. A controller of the microscope can turn on light sources configured to generate a fluorescence excitation signal, so that fluorescence signals emitted by the beads can be captured by the cameras having the filters. The process can continue for other filters to capture fluorescence images under different fluorescence excitation signals. From the fluorescence images, the barcode of each bead can be determined, and related to the cell that is co-isolated in the well in order to create a link between the spatial identity (start and end location) of each cell and its barcode identity (sequencing readout/molecular information). For example, the association of the barcode information in the beads to the well locations in the well plate can be used as a part of the tracking the molecular information resulted from the sequencing to the original cell positions in the sample, through the barcode information to the well locations to the cells in the identified wells, and to the original cells in the sample.

In some embodiments, in-situ-sequencing-by-synthesis could be used as a complementary or alternative approach to barcoded beads.

FIG. 17D shows a lysis process, for example, by introducing a lysis solution 1715 to the well plate. The membranes of the cells can be destroyed to expose the content of the cells, such as the molecules in the cells. The exposed molecules can be bound to the oligos of the beads. Excess lysis solution can be drained 1715A. In some embodiments, the lysis process can be performed before or after the bead identification process.

FIG. 17E shows a collection of the cells from the wells. With the barcode information, the sequencing can distinguish between beads to allow the cells to undergo a sequencing process together, instead of individually. The sequencing process for multiple cells can significantly save time and expenses.

FIG. 17F shows a process 1737 for a preparation of a sequence library and the sequencing process. FIG. 17G shows a process 1731 for analysis, extract genomes, epigenomes, transcriptomes, proteins, and then forming a spatial reconstruction of the sample using the barcode information and the movement tracking of the cell movements to identify the original positions of the cells in the sample.

In some embodiments, the spatial biology process using barcode beads coupled to the cell contents has a first portion similar to the process of analyzing single cell. The barcode process can start with a sample preparation and capturing images of the sample with high resolution (shown previously). The sample then is disposed in an assembly fixture to perform cell dissociation, such as with an enzyme, cell movements to wells of a well plate, and cell distribution at wells. The assembly fixture is disposed in a microscope having multiple cameras to provide a field of view large enough to cover the sample and the movements of cells, together with a resolution high enough to observe the individual cells and their movements and low enough to be able to track the cell movements, e.g., a fast camera capture speed to track the movements of all cells, especially when the cells crossing paths.

The cells in the wells can be analyzed separately, or can be mixed together for a faster analysis. In the case of cell mixing, a process for uniquely identifying cells can be used to distinguish the molecular information obtained from the analysis of the mixed cells. An in-situ sequence-by-synthesis approach or a barcoding process can be used. The barcoding process is shown in detail, but other cell identification process in mixed cell analysis can be used.

For example, barcode beads can be placed in a solution and the solution can be introduced to the assembly fixture, such as to the well plate, so that the beads can enter the wells by settling into position with gravity. The beads can be introduced to the well plate after the dissociation of the sample, e.g., after the cells have been dissociated from the sample and travel to the well plate to enter the wells. Alternatively, the beads can be introduced to the well plate before the dissociation of the sample, e.g., before the cells are dissociated from the sample. After the beads are settled in the wells, as observed and characterized by the multiple cameras of the microscope, the cells can be dissociated from the sample to enter the wells. In some embodiments, the wells can be designed with optimal shapes, diameters and depths to promote co-isolation of a single cell and a single bead.

After the cells and the beads are settled in the wells, the cells can be lysed, for example, by introducing a lysis solution to the assembly fixture, such as to the well plate. The lysis process can destroy the membranes of the cells to expose the content of the cells, allowing the molecules in the cells to bind with the oligos of the beads. For example, the mRNA from the cells can bind to the synthetic oligos at a universal binding site of the oligos. Alternatively, the cells can be lysed before the beads are introduced to the wells.

Barcoding the content of the individual cells can allow the assigning of a unique signature to a cell (or the RNA in a cell), which can allow the mixing of the cells in a pool for batch processing with next-generation sequencing. For example, the sequencing process can provide the barcode information, which is unique to each bead, and the molecular information. Thus, the molecular information from a pool of cells can be grouped into separate individual beads. The beads can also carry other identifiers, such as unique molecular identifier, which can allow the differentiation and the quantification of individual molecules within the cells.

The sequencing process only relates the molecular information to the barcodes of the beads, and thus a correlation between the bead barcodes and the cells, or the wells in which the cells are located, is needed.

In some embodiments, the barcode of each bead in a well can be determined and related to the cell that is co-isolated in the well. A sequential in-situ sequencing multi-channel fluorescence process can be performed, for example, by the microscope equipped with fluorescence filters.

The molecular information resulted from the sequencing process can be correlated with the cells in their original positions in the sample, through the barcode identities from the sequencing readouts, traced to the well locations in the well plate, and then traced to the original positions of the cells through the start and end locations of the cell movements tracked by the microscope.

In some embodiments, assays can be run on a group of cells, including cell sequencing. This information can be saved on a per-cell basis since each cell is distinguishable and isolated in individual wells. The aforementioned barcoding approach can then be used such that each of these wells has a bead with a unique barcode that is associated with its position on the well plate. Molecular contents of each cell may be attached to oligonucleotides on the bead, each oligo having the same positional barcode element that identifies it with a particular well. All of the oligos on all of the beads may be mixed together and sequenced on an Illumina sequencer, for example. And since each oligo has a location-identifying barcode, the molecular data for the oligos may be attached to the well location and the well location is likewise attached to the original cell location. A database can be used to track each cell's identity, location in the well, and location in the original specimen. Thus, spatial information as to the cells' original location in the specimen can be known for each cell and its associated molecular data which are tied together via the unique barcode for the location in the well plate, together with the cell movement tracking to the cell original positions in the sample.

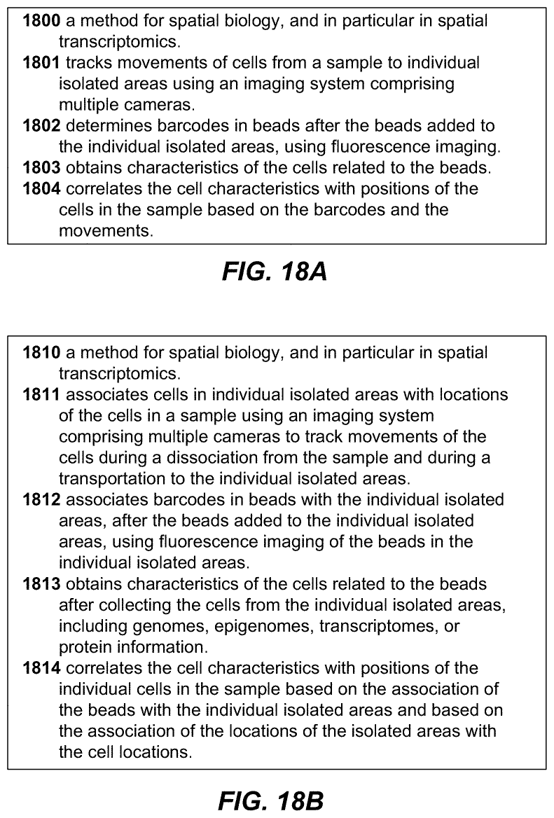

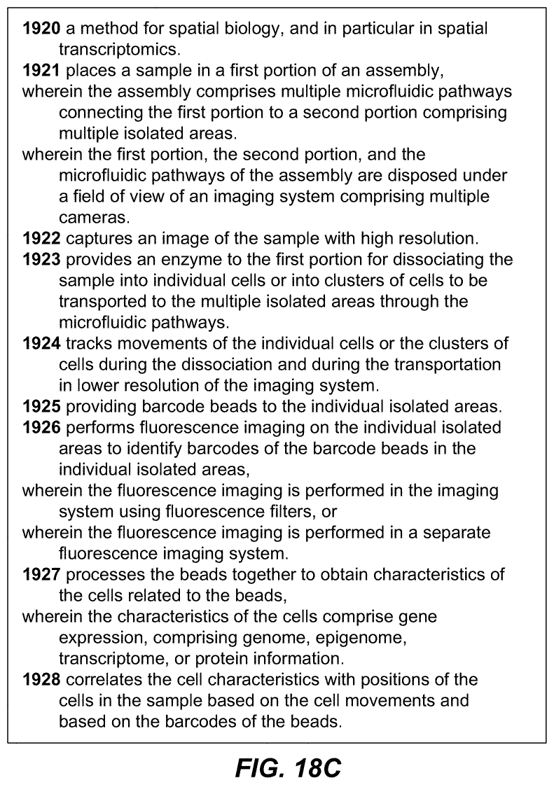

FIGS. 18A-18C illustrate flow charts for a spatial construction of molecular information of cells in a sample according to some embodiments. In FIG. 18A, operation 1800 discloses a method for spatial biology, and in particular in spatial transcriptomics. Operation 1801 tracks movements of cells from a sample to individual isolated areas using an imaging system comprising multiple cameras. Operation 1802 determines barcodes in beads after the beads added to the individual isolated areas, using fluorescence imaging. Operation 1803 obtains characteristics of the cells related to the beads. Operation 1804 correlates the cell characteristics with positions of the cells in the sample based on the barcodes and the movements.

In FIG. 18B, operation 1810 discloses a method for spatial biology, and in particular in spatial transcriptomics. Operation 1811 associates cells in individual isolated areas with locations of the cells in a sample using an imaging system comprising multiple cameras to track movements of the cells during a dissociation from the sample and during a transportation to the individual isolated areas. Operation 1812 associates barcodes in beads with the individual isolated areas, after the beads added to the individual isolated areas, using fluorescence imaging of the beads in the individual isolated areas. Operation 1813 obtains characteristics of the cells related to the beads after collecting the cells from the individual isolated areas, including genomes, epigenomes, transcriptomes, or protein information. Operation 1814 correlates the cell characteristics with positions of the individual cells in the sample based on the association of the beads with the individual isolated areas and based on the association of the locations of the isolated areas with the cell locations.

In FIG. 18C, operation 1820 discloses a method for spatial biology, and in particular in spatial transcriptomics. Operation 1821 places a sample in a first portion of an assembly fixture. The assembly fixture comprises multiple microfluidic pathways connecting the first portion to a second portion comprising multiple isolated areas. The first portion, the second portion, and the microfluidic pathways of the assembly fixture are disposed under a field of view of an imaging system comprising multiple cameras. Operation 1822 captures an image of the sample with high resolution.

Operation 1823 provides an enzyme to the first portion for dissociating the sample into individual cells or into clusters of cells to be transported to the multiple isolated areas through the microfluidic pathways. Operation 1824 tracks movements of the individual cells or the clusters of cells during the dissociation and during the transportation in lower resolution of the imaging system. Operation 1825 provides barcode beads to the individual isolated areas. Operation 1826 performs fluorescence imaging on the individual isolated areas to identify barcodes of the barcode beads in the individual isolated areas. The fluorescence imaging is performed in the imaging system using fluorescence filters, or the fluorescence imaging is performed in a separate fluorescence imaging system.

Operation 1827 processes the beads together to obtain characteristics of the cells related to the beads. The characteristics of the cells comprise gene expression, comprising genome, epigenome, transcriptome, or protein information. Operation 1828 correlates the cell characteristics with positions of the cells in the sample based on the cell movements and based on the barcodes of the beads.

In some embodiments, the reference numbers are classified with the last 2 digits referring to a same component or element, and the first one or 2 digits referring to the number of the figures. For example, 101, 301, 401, 501, 601, 901, 1001, 1201, 1501, 1601, and 1701 all refer to camera, either a camera, multiple cameras, or a camera array, e.g., the last 2 digits of 01 refer to a camera, while the first one or two digits of 1, 3, 4, 5, 6, 9, 10, 12, 15, 16, and 17 refer to the figure numbers. There can be exception, for example, in the flow chart number for the steps.

What is claimed is:

1. A method for spatial biology, the method comprising
tracking movements of individual cells in a sample during a dissociation from the sample and during a transportation to individual isolated areas using an imaging system,
analyzing the individual cells for information comprising at least one of molecular information or cellular information;
correlating the information of the individual cells with positions of the individual cells in the sample based on the cell movements.

2. A method as in claim 1,
wherein the positions of the individual cells in the sample are determined by tracing back the movements of the individual cells from ends of the movements at the individual isolated areas to beginnings of the movements.

3. A method as in claim 1, further comprising
spatially constructing a map of the information of the sample based on position of the multiple cells.

4. A method as in claim 1,
wherein analyzing the multiple cells comprises analyzing one or more cells in each isolated area separately, or
wherein analyzing the multiple cells comprises analyzing cells in the multiple isolated areas together after adding barcode beads to the multiple isolated areas for sorting the individual cells based on the barcode beads.

5. A method as in claim 1,
keeping a viable environment for the cells during the dissociation and the transportation for the analysis.

6. A method comprising
placing a sample in an assembly fixture in a microscope,
wherein the assembly fixture comprises a first section configured for accepting the sample,
wherein the assembly fixture comprises a second section comprising multiple isolated areas,
wherein the microscope comprises multiple cameras comprising a combined field of view to the assembly fixture;
tracking movements of multiple cells of the sample from the first section to the multiple isolated areas;
analyzing the multiple cells for information comprising at least one of molecular information or cellular information;
correlating the information of each cell of the multiple cells with a position of the each cell in the sample based on the movements of the each cell.

7. A method as in claim 6, further comprising at least one of
delivering an enzyme solution to at least the first section for dissociating the sample into the multiple cells;
delivering a lysis solution to at least the second section for distributing into the multiple isolated areas; or
delivering a solution comprising barcode beads to at least the second section for distributing into the multiple isolated areas.

8. A method as in claim 6, further comprising
setting the imaging system to a high resolution with the high resolution configured for obtaining positions of the cells in the sample before the movements of the cells,
setting the imaging system to a lower resolution with the lower resolution configured for real time tracking the movements of the cells in the sample.

9. A method as in claim 6,
wherein the position of the each cell in the sample is determined by tracing back the movements of the each cell from an end of the movements at an isolated area to a beginning of the movements.

10. A method as in claim 6, further comprising
spatially constructing a map of the information of the sample based on position of the multiple cells.

11. A method as in claim 6, further comprising
delivering barcode beads to the second section for distributing into the multiple isolated areas,
capturing fluorescence signals from the barcodes of the barcode beads in each isolated area of the multiple isolated areas using the image system with fluorescence filters for identifying the barcodes.

12. A method as in claim 6,
wherein the assembly fixture comprises flow cytometers coupled to the first section to provide improvements on cell sorting, cell counting, cell fluorescence data gathering, or spatial cell information retaining after an analysis on the cells.

13. A method as in claim 6,
wherein analyzing the multiple cells comprises analyzing cells in the multiple isolated areas together and using barcode beads for separating the individual cells.

14. A method as in claim 6,
correlating the information of the each cell of the multiple cells with a barcode of a bead in a corresponding isolated area based on bead barcodes identification, and then with a position of the each cell in the sample based on the movements of the each cell.

15. A microscope comprising
multiple cameras.
wherein each camera of the multiple cameras is configured to capture one or more images of a region of an assembly fixture,
wherein the assembly fixture comprises a first section configured to accept a sample, and a second section comprising multiple isolated areas,
wherein a combine field of view of the multiple cameras is configured to cover at least portions of the first and second sections of the assembly fixture for capturing images of movements of multiple cells of the sample from the first section to the multiple isolated areas;
one or more radiation sources.
wherein the one or more radiation sources are configured to illuminate the sample;
a processor.
wherein the processor is configured to control the one or more radiation sources to create one or more illumination patterns to the sample.
wherein the processor is configured to control the multiple cameras to capture images of the sample under the one or more illumination patterns.
wherein the processor is configured to track the movements of the multiple cells during a dissociation from the sample and during a transportation to the multiple isolated areas.

16. A microscope as in claim 15,
wherein the assembly fixture comprises an input for accepting an enzyme solution, a lysis solution, or a solution comprising barcode beads,
wherein the assembly fixture comprises microfluidic conducts for directing the multiple cells from the first section to the second section, with a first microfluidic conduit of the microfluidic conducts configured for sheering clusters of cells from the first section to the second section and a second microfluidic conduit of the microfluidic conducts comprising filters for blocking debris,
wherein the second section of the assembly fixture is configured to accept a microwell plate comprising the multiple isolated areas of microwells, or comprises multiple isolated areas of microwells,
wherein the assembly fixture is at least partially transparent with respect to radiation from the sample for the multiple cameras to track the movements of the multiple cells in the assembly fixture.

17. A microscope as in claim 15,
wherein the multiple cameras comprise a high resolution setting configured for obtaining positions of the cells in the sample before the movements of the cells,
wherein the multiple cameras comprise a lower resolution setting for real time tracking the movements of the cells in the sample.

18. A microscope as in claim 15,
wherein the imaging system comprises a moving mechanism configured to move a filter assembly fixture between multiple positions,
wherein in different positions of the multiple positions, the filter assembly fixture is configured to provide different filters to a camera of the multiple cameras for capturing different fluorescence signals from the assembly fixture.
wherein at least a camera of the multiple cameras comprises a fluorescence filter configured to capture a fluorescence signal from the assembly fixture.

19. A microscope as in claim 15,
wherein the microscope further comprises multiple filters configured for sequentially capturing fluorescence signals generated from barcodes of barcode beads added to the multiple isolated areas,
wherein the processor is configured to generate a spatial map of molecular information of each cell of the multiple cells in the sample based on a barcode identification in a corresponding isolated area of the each cell, and then with a position of the each cell in the sample based on the movements of the each cell.

20. A microscope as in claim 15,
wherein the processor is configured to generate a spatial map of molecular information of the multiple cells in the sample based on position of the multiple cells determined from the movement tracking.
wherein the position of the each cell in the sample is determined by finding a beginning of the movements of the each cell.
